(19) United States
(12) Patent Application Publication
(10) Pub. No.: US 2005/0277192 A1
(43) Pub. Date: Dec. 15, 2005

(54) Hepatitis C virus nonstructural protein 4A (NS4A) is an enhancer element

(76) Inventor: Matti Sallberg, Alvsjo (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/137,220

(22) Filed: May 24, 2005

Related U.S. Application Data
(63) Continuation of application No. PCT/IB03/06488, filed on Nov. 25, 2003.
(60) Provisional application No. 60/430,009, filed on Nov. 26, 2002.

(51) Int. Cl. C12N 15/86

(52) U.S. Cl. 435/456

(57) ABSTRACT

Aspects of the present invention concern the discovery of an enhancer that regulates the expression of an associated gene. More particularly, it was found that the nonstructural protein 4A (NS4A) from the hepatitis C virus (HCV) modulates the expression and immunogenicity of an associated nucleic acid.

### CROSS-REFERENCE TO RELATED APPLICATIONS

This application is a continuation of international application number PCT/IB2003/006488, and claims the benefit of priority of international application number PCT/IB2003/006488 having international filing date of Nov. 25, 2003, designating the United States of America and published in English, which claims the benefit of priority of U.S. provisional patent application No. 60/430,009, filed Nov. 26, 2002; both of which are hereby expressly incorporated by reference in their entireties.

### FIELD OF THE INVENTION

The present invention concerns the discovery of an enhancer that regulates the expression of an associated gene. More particularly, it was found that the nonstructural protein 4A (NS4A) from the hepatitis C virus (HCV) modulates the expression and immunogenicity of an associated nucleic acid.

### BACKGROUND OF THE INVENTION

Enhancers are cis-acting elements that increase the level of transcription of an adjacent gene from a promoter. Oftentimes, the enhancement of transcription is relatively independent of the position and orientation of the enhancer element. (See Khoury and Gruss, Cell 33:313 (1983)). Enhancer elements have been identified in a number of viruses, including polyoma virus, papilloma virus, adenovirus, retrovirus, hepatitis virus, cytomegalovirus, herpes virus, papovaviruses, such as simian virus 40 (SV40) and BK, and in many non-viral genes, such as within mouse immunoglobulin gene introns. (See e.g., U.S. Pat. No. RE37,806, herein expressly incorporated by reference in its entirety). Enhancers that operate in mammalian cells are particularly useful in biotechnology, immunology, and medicine and the need for more enhancers is manifest.

### SUMMARY OF THE INVENTION

It was discovered that hepatitis C virus (HCV) nonstructural protein 4A (NS4A) enhances the transcription and immunogenicity of an associated nucleic acid. In a first set of experiments it was observed that when HCV-1 NS3/4A gene was transfected into mammalian cells, vis a vis a eukaryotic expression vector, the expression level of NS3 was higher than when the HCV-1 NS3 gene and expression vector were transfected alone (i.e., without NS4A). Further, mice immunized with the NS3/4A gene were found to have primed 10 to 100-fold higher levels of NS3-specific antibodies, as compared to mice immunized with the NS3 gene alone. The humoral responses primed by the NS3/4A gene exhibited a higher IgG2a/IgG1 ratio (>20) as compared to the NS3 gene (3.0), providing evidence of a T helper 1-skewed response.

In another set of experiments, it was discovered that when mice carrying NS3/NS4A expressing SP2/0 myeloma cells were immunized i.m. with a low dose of the NS3/4A gene (10 μg), the growth of NS3/4A-expressing tumor cells was inhibited; whereas low dose immunization of the mice with the NS3 gene alone or NS3 protein provided no inhibition of growth of the NS3/4A expressing tumor cells. Further, it was determined that when a gene gun was used, only three 4 μg doses of the NS3/4A gene were required to efficiently prime cytotoxic T lymphocyte (CTL) responses at a precursor frequency of 2% to 4% and to inhibit the growth of NS3/4A-expressing tumor cells in mice carrying NS3/NS4A expressing SP2/0 myeloma cells.

Several embodiments of the invention include approaches to enhance the level of transcription of a nucleic acid that is associated with NS4A. In some methods, for example, the expression of a nucleic acid in a cell is increased or enhanced by providing a first nucleic acid encoding an hepatitis C virus (HCV) non-structural protein 4A (NS4A) or functional portion thereof, identifying a second nucleic acid for enhanced expression; and associating said second nucleic acid with said first nucleic acid in said cell, whereby such association results in an enhanced expression of said second nucleic acid. In some applications, the second nucleic acid is an HCV non-structural protein 3 (NS3). The first and second nucleic acid can be joined in cis, juxtaposed, on the same construct, on separate constructs, or in trans. Additionally, in some applications, the first nucleic acid consists of between 10 and 20, between 20 and 30, between 30 and 40, or between 50 and 54 consecutive amino acids of SEQ. ID. NO. 2.

More embodiments of the invention concern approaches to enhance the inmmunogenicity of a nucleic acid that is associated with NS4A. In some methods, for example, the immunogenicity of a nucleic acid is increased or enhanced by providing a first nucleic acid encoding an hepatitis C virus (HCV) non-structural protein 4A (NS4A) or functional portion thereof, identifying a second nucleic acid for enhanced immunogenicity, and associating said second nucleic acid with said first nucleic acid, whereby such association results in an enhanced immunogenicity of said second nucleic acid. In some applications, the second nucleic acid is an HCV non-structural protein 3 (NS3). The first and second nucleic acid can be joined in cis, juxtaposed, on the same construct, on separate constructs, or in trans. Additionally, in some applications, the first nucleic acid consists of between 10 and 20, between 20 and 30, between 30 and 40, or between 50 and 54 consecutive amino acids of SEQ. ID. NO.2.

### BRIEF DESCRIPTION OF THE DRAWINGS

FIG. 1 In vitro translation products created from the plasmids NS3-pVAX1, NS3/4A-pVAX1, and mNS3/4A-pVAX1 in the presence of ³⁵S-methionine and resolved by SDS-PAGE. Lane 1, Molecular weight marker (CFA 756; Amersham Pharmacia Biotech); lane 2, 61 kDa kit control, lane 3, negative control, lane 4, NS3-pVAX1, lane 5, NS3/4A-pVAX1, and lane 6, mNS3/4A-pVAX1.

FIG. 2. Analysis of the NS3 protein expressed by rSFV-NS3 (a), mNS3/4a (b), or NS3/4a (c) infected BHK-21 cells. After labelling with 35S methionine, cells were “chased” with cold methionine for the indicated times. The resulting cell lysates were analysed by immunoprecipitation and 10% SDS PAGE. NS3 expression was also analyzed in rSFV-NS3 (d) and rSFV-NS3/4A (e) infected BHK cells by immunofluorescent staining, using an NS3-specific monoclonal antibody. Cells were stained 24 hours after infection and a greater dispersion of the NS3 protein in rSFV-NS3 infected cells (e) was observed.

FIG. 3. Antibody responses primed by immunizations with 100 μg NS3-pVAX1 or NS3/4A-pVAX1 in groups of five H-2^{d} mice (a-b). Arrows indicate time point of immunization. All mice were pre-treated with cardiotoxin. Values are given as mean end-point antibody ±SD. A comparison of the humoral responses primed by 100 μg NS3-pVAX1, NS3/4A-pVAX1, or mNS3/4A-pVAX1 in groups of ten to twenty H-2^{d} mice is also shown. Mice were primed and boosted at week 0 and 4. Values are given as mean end-point antibody titre ±SD. A solid line indicates a significant difference of p<0.01, a broken line a difference of p<0.05, and a dotted line indicates that no significant difference (Mann-Whitney U-test) was observed.

FIG. 4. T cell responses to NS3 in spleens from immunized H-2^{d} mice. Groups of five mice were immunized with 100 μg NS3-pVAX1 or NS3/4A-pVAX1. All mice were pre-treated with cardiotoxin. Values are given as the antigen-induced proliferation minus the spontaneous proliferation (Δcpm). Values are shown as mean cpm values ±SD of triplicate determinations (a). Comparison of the NS3-specific IgG subclass response at week six in BALB/c mice immunized with rNS3 (20 μg) in PBS, NS3-pVAX1 or NS3/4A-pVAX1 (b). Values have been given as the mean end point titre ±SD of IgG1 or IgG2a antibodies to NS3. The titer ratios were obtained by dividing the mean endpont titer of IgG2a antibodies to NS3 by the mean endpont titer IgG1 antibodies to NS3. A high ratio (>3) indicates a Th1-like response and a low ratio (<0.3) indicates a Th2-like response, whereas values within a three-fold difference from 1 (0.3 to 3) indicates a mixed Th1/Th2 response. Also given (c) are the proliferative responses in the spleen after one immunization with rNS3 in CFA, after three monthly injections with the NS3/4A-pVAX1 plasmid given i.m. (these mice were sacrificed six weeks after the last injection). Values are shown as mean cpm values of triplicate determinations (c).

FIG. 5. Kinetics of the priming of in vitro detectable CTLs in H-2^{d} mice. Groups of five H-2^{d} mice were immunized i.m. with 100 μg NS3/4A-pVAX1 at monthly intervals. All mice were pre-treated with cardiotoxin. Results from the cytotoxicity assays have been given from two injections (a), three injections (b), and six injections of 100 μg DNA (c). The percent specific lysis corresponds to the percent lysis obtained with NS3/4A expressing SP2/0 cells minus the percent lysis obtained with non-transfected SP2/0 cells. Values have been given for effector to target (E:T) cell ratios of 40:1, 20:1 and 10:1. More than 10% specific lysis was considered as positive. Each line corresponds to an individual mouse.

FIG. 6. Inhibition of tumor cell growth in vivo using different modes of immunization. Groups of five to ten H-2^{d} mice were immunized with either PBS or 20 μg rNS3 in CFA given i.p. or 100 μg of control plasmid (p17-pcDNA3) (a) or with 10 μg of NS3-pVAX1 or NS3/4A-pVAX1 (b) or 100 μg of NS3-pVAX1 or NS3/4A-pVAX1 or mNS3/4A-pVAX1 (c). Mice were primed and boosted at week 4, 8, 12 and 16. All mice were pre-treated with cardiotoxin. Two weeks after last immunization, mice were injected with 2×10⁶ NS3/4A-expressing SP2/0 cells s.c. The size of each tumor was measured through the skin at days seven, 11 and 13 after tumor injection. Mean tumour growth in each group was assessed for the whole period and groups were compared statistically using area under the curve (AUC) and ANOVA. In (d), the statistical comparisons between the experimental groups and the control groups is provided.

FIG. 7. Histological appearance of solid tumors excised from non-immunized mice (a and b), mice immunized with 10 μg NS3/4A-pVAX1 (c and d), and mice immunized with 100 μg NS3/4A-pVAX1 (e and f). Sections of NS3/4A expressing SP2/0 myeloma stained by Hematoxylin-Eosin (a, c, and e) or by anti-CD3 antibody (b, d, and f). The insert in figure (a) shows the results from testing the transfected cell line for expression of NS3/4A mRNA by RT-PCR. Lanes 1 and 2 shows the molecular weight markers, lane 3 the NS3/4A-SP2/0 cells, lane 4 the SP2/0 cells, lanes 5, 7, and 8 are negative controls, and line 6 a DNA PCR of the NS3/4A-pVAX1 plasmid giving a band of 2,061 bases.

FIG. 8. Gene gun immunization with NS3/4A-pVAX1 induces CTL specific for a H-2D^{b}-restricted peptide epitope. Groups of five to ten C57BL/6 mice were immunized s.c. with 100 μg NS3-specific peptide (GAVQNEVTL (SEQ. ID. No. 1)) in CFA or transdermally with 4 μg DNA/dose using the gene gun at monthly intervals. Spleen cells from naive (a) or NS3/4A peptide immunized mice (b) or NS3/4A-pVAX1 gene gun immunized mice (d) were restimulated 5 days in vitro with irradiated NS3-peptide loaded naive spleen cells. Spleen cells from gene gun immunized mice restimulated with an irrelevant H-2D^{b} binding peptide served as negative control (c). In panel d) white boxes indicates the % specific lysis after three immunizations and black boxes represent the % specific lysis after four immunizations. Within the parentheses the peptide used in the restimulation cultures have been indicated. Each line represents data from an individual mouse.

FIG. 9. Induction of NS3/4A-specific CD8 T cells after gene gun immunization. The frequency of NS3/4A peptide specific CD8 T cells were determined by flow cytometric staining of spleen cells from naive mice (a, c, e, and g) and NS3/4A-pVAX1 DNA immunized mice (b, d, f and h) with dimeric H-2D^{b}:Ig fusion protein loaded with the NS3 peptide (GAVQNEVTL (SEQ. ID. No. 1)). Unloaded H-2D^{b}:Ig fusion protein was used to monitor unspecific staining (g and h). A total of 150,000-200,000 cells were collected and the percentage of CD8+ cells stained for H-2D^{b}:Ig are indicated in the parentheses in each dot-plot.

FIG. 10. Inhibition of tumor growth in vivo using gene gun immunization. Groups of ten BALB/c mice were either left untreated or were given four monthly transdermal immunizations with a 4 μg DNA/dose of NS3/4A-pVAX1. Four weeks after the last immunization, the mice were injected s.c with 1×10⁶ NS3/4A-expressing SP2/0 cells. Tumor sizes were measured through the skin at days 6, 7, 8, 10, 11, 12, 13, and 14, 15 after tumor injection. The area under the curve for the two curves was statistically different (ANOVA; p <0.01).

FIG. 11. Priming of in vitro detectable CTLs in H-2^{b} mice by gene gun immunization of the wtNS3-pVAX1 (wild-type NS3), wtNS3/4A (wild-type NS3/4A), and coNS3/4A (human codon-optimized NS3/4A) plasmids, or s.c. injection of wtNS3/4A-SFV particles (NS3/4A containing Semliki Forest virus particles). Groups of five to 10 H-2^{b} mice were immunized once (a) or twice (b). The percent specific lysis corresponds to the percent lysis obtained with either NS3-peptide coated RMA-S cells (upper panel in a and b) or NS3/4A-expressing EL-4 cells (lower panel in a and b) minus the percent lysis obtained with unloaded or non-transfected EL-4 cells. Values have been given for effector to target (E:T) cell ratios of 60:1, 20:1 and 7:1. Each line indicates an individual mouse.

FIG. 12. Evaluation of the ability of different immunogens to prime HCV NS3/4A-specific tumor-inhibiting responses after a single immunization. Groups of ten C57BL/6 mice were either left untreated or were given one immunization with the indicated immunogen, as described in FIG. 11, (4 μg DNA using gene gun in (a), (b), (c), (g), and (h); 10⁷ SFV particles s.c. in (d); 100 μg peptide in CFA s.c. in (e); and 20 μg rNS3 in CFA s.c. in (f). Two weeks after the last immunization, the mice were injected s.c with 106 NS3/4A-expressing EL-4 cells. Tumor sizes were measured through the skin at days 6 to 19 after tumor injection. Values have been given as the mean tumor size +standard error. In (a) to (e), as a negative control, the mean data from the group immunized with the empty pVAX plasmid by gene gun has been plotted in each graph. In (f) to (h) the negative controls were non-immunized mice. Also given is the p value obtained from the statistical comparison of the control with each curve using the area under the curve and ANOVA.

### DETAILED DESCRIPTION OF THE INVENTION

It has been discovered that the NS4A gene from hepatitis C virus (HCV) is an enhancer that increases transcription and immunogenicity of an associated gene or nucleic acid. Data provided herein demonstrate that when HCV-1 NS3/4A gene was transfected into mammalian cells, vis a vis a eukaryotic expression vector, the expression level of NS3 was higher than when the HCV-1 NS3 gene and expression vector were transfected alone (i.e., without NS4A). Mice immunized with the NS3/4A gene were found to have primed 10 to 100-fold higher levels of NS3-specific antibodies, as compared to mice immunized with the NS3 gene alone. NS3-specific cytotoxic T lymphocyte (CTL)s were effectively primed by the NS3/4A-pVAX1 plasmid administered i.m. or transdermally. Further, four gene gun immunizations with 4 μg plasmid per dose elicited a potent immune response, wherein approximately 4% of the total splenic CD8+ population were NS3/4A-specific T cells. These responses were active in vivo and were sufficient to inhibit the growth of NS3/4A-expressing tumor cells. When administered transdermally at doses commensurate with immunogen doses used in human clinical trials, the NS3/4A-pVAX1 immunogen was found to be very effective in priming NS3-specific CTLs.

Embodiments described herein concern the use of genetic constructs comprising the NS4A enhancer to increase the transcription or immunogenicity of an associated nucleic acid (e.g., a gene encoding NS3). Expression constructs comprising the NS4A enhancer can be used, for example, to enhance the expression of a marker gene (e.g., Green Fluorescent Protein or “GFP,” or lac Z), a nucleic acid encoding an immunogen (e.g., a hepatitis or HIV antigen), or a therapeutic nucleic acid (e.g., an antisense construct). Expression constructs comprising the NS4A enhancer can also be formulated to be the active ingredient in vaccines and compostions that are used to generate an immune response to an associated gene or gene product. Preferred embodiments employ compositions that are formulated for gene gun delivery, which comprise any amount between about 0.1-20 μg of an expression construct that comprises the NS4A enhancer or functional portion thereof and an associated gene. (e.g., 0.1 μg, 0.5 μg, 1 μg, 3 μg, 5 μg, 7 μg, 10 μg, 13 μg, 15 μg, 17 μg, or 20 μg).

The methods described herein can be practiced by providing a cell, preferably a cell that exists in a mammal (e.g., human, cat, dog, horse, and sheep) or a plant, with an amount of a composition comprising the NS4A enhancer that is sufficient to increase the expression of a subject gene that is joined to said the NS4A enhancer. The examples provided in the following sections demonstrate that the enhancer activity (e.g., upregulation of transcription of an associated gene and increased immunogenicity to said associated gene and/or gene product) occurs in both cell culture (in vitro) and in mammals (in vivo).

The section below describes the NS4A enhancer and constructs containing the NS4A enhancer.

The NS4A Enhancer

Hepatitis C Virus (HCV) belongs to the Flaviviridae family of single-stranded RNA viruses. (Virology, Fields ed., third edition, Lippencott-Raven publishers, pp 945-51 (1996)). The HCV genome is approximately 9.6 kb in length, and encodes at least ten polypeptides. (Kato, Microb. Comp. Genomics, 5:129-151 (2000)). The genomic RNA is translated into one single polyprotein that is subsequently cleaved by viral and cellular proteases to yield the functional polypeptides. (Id.) The polyprotein is cleaved to three structural proteins (core protein, E1 and E2), to p7 of unknown function, and to six non-structural (NS) proteins (NS2, NS3, NS4A/B, NS5A/B). (Id.) NS3 encodes a serine protease that is responsible for some of the proteolytic events required for virus maturation (Kwong et al., Antiviral Res., 41:67-84 (1999)) and NS4A acts as a co-factor for the NS3 protease. (Id.) NS3 further displays NTPase activity, and possesses RNA helicase activity in vitro. (Kwong et al., Curr. Top. Microbiol. Immunol., 242:171-96 (2000)).

The importance of using NS3/4A as an immunogen has been recognized. See e.g., U.S. application Ser. No. 09/929,955 and U.S. application Ser. No. 09/930,591, both of which are hereby expressly incorporated by reference in their entireties. Further, humoral response to a genetic immunogen containing the complete NS3/4A protease is surprisingly strong. (Lazdina et al., J Gen Virol, 82:1299-1308 (2001)). The reason for this was not evident at the time, though some conjectured that the presence of the cofactor NS4A increased the intracellular stability of NS3. (Wolk et al., J Virol, 74:2293-2304 (2000) and Tanji et al., J Virol, 69:1575-1581 (1995)). The increase in stability hypothesis was supported by the fact that the amino terminal domain of NS4A targeted the NS3/4A complex to intracellular membranes. (Tanji et al., J Virol, 69:1575-1581 (1995)). Additionally, both the protease and helicase activities of HCV NS3 require the presence of NS4A. (Bartenschlager et al., J Virol, 67:3835-3844 (1993); Bartenschlager et al., J Virol, 69:7519-7528 (1995); Failla et al., J Virol, 68:3753-3760 (1994); Pang et al., Embo J, 21:1168-1176 (2002)). Until the present disclosure, however, it had not been realized that the NS4A gene and portions thereof enhance the expression and immunogenicity of an associated gene.

The term “NS4A enhancer” refers to any NS4A gene from any HCV isolate (preferably HCV-1b) that enhances the transcription of an associated nucleic acid and/or the immunogenicity of said associated nucleic acid. In some contexts, the term “NS4A enhancer” refers to a portion of an NS4A gene of an HCV isolate (preferably HCV-1) that retains the ability to increase transcription of an associated gene and/or the immunogenicity to said associated gene. That is, an “NS4A enhancer” can consist, consist essentially of, or comprise a nucleic acid that encodes any amount between about 3-54 consecutive amino acids of NS4A (e.g., STWVLVGGVL AALAAYCLTT GSVVIVGRII LSGKPAIIPD REVLYREFDE MEEC (SEQ. ID. NO. 2), as disclosed by accession number CAB46677 and Lohmann et al., Science 285:110-113 (1999), herein expressly incorporated by reference in its entirety) so long as the molecule retains the ability to increase transcription of an associated gene and/or immunogenicity to said associated gene. That is, the NS4A enhancer can consist, consist essentially of, or comprise a nucleic acid that encodes at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 consecutive amino acids of NS4A (SEQ. ID. NO. 2).

Additionally, an “NS4A enhancer” can consist, consist essentially of, or comprise a nucleic acid that is any amount between about 9-162 consecutive nucleotides of an NS4A gene (e.g., AGCACCTGGG TGCTGGTAGG CGGAGTCCTA GCAGCTCTGG CCGCGTATTG CCTGACAACA GGCAGCGTGG TCATTGTGGG CAGGATCATC TTGTCCGGAA AGCCGGCCAT CATTCCCGAC AGGGAAGTCC TTTACCGGGA GTTCGATGAG ATGGAAGAGT GC (SEQ. ID. NO. 3), as disclosed by accession number AJ238799 and Lohmann et al., Science 285:110-113 (1999), herein expressly incorporated by reference in its entirety) so long as the molecule retains the ability to increase transcription of an associated gene and/or immunogenicity to said associated gene. That is, an NS4A enhancer can consist, consist essentially of, or comprise a nucleic acid, which is at least 9, 15, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, or 162 consecutive nucleotides of an NS4A gene (e.g., SEQ. ID. NO. 3).

The NS4A enhancers described herein can be incorporated into genetic constructs, e.g., expression constructs, that are designed such that any desired subject nucleic acid to be enhanced (e.g., NS3) can be associated with the NS4A enhancer. Such associations can be in “cis”, which is on the same plasmid, or in “trans,” which is on separate plasmids. Preferably, the NS4A and the nucleic acid to be enhanced are juxtaposed. Desirably, such constructs have convenient restriction sites (e.g., a multiple cloning site) at or near the NS4A enhancer that allows for the subject nucleic acid to be easily inserted in a cassette-like fashion and joined to the NS4A enhancer.

The example below describes the manufacture of several constructs, which were used to identify and characterize the NS4A enhancer.

### EXAMPLE 1

Constructs containing NS3 and NS3/4A genes were created as follows. A full-length NS3 and NS3/NS4A gene fragment was amplified from a patient infected with HCV genotype 1b, as previously described. (Lazdina et al., J Gen Virol, 82:1299-1308 (2001), herein expressly incorporated by reference in its entirety). The NS3 and NS3/4A genes were inserted into the eukaryotic expression vector pVAX1 (Invitrogen, San Diego, Calif.). For amplification of NS3, the forward primer 5′-GTG GAA TTC ATG GCG CCT ATC ACG GCC TAT-3′ (SEQ. ID. NO. 4), and reverse primer 5′-CCA CGC GGC CGC GAC GAC CTA CAG-3′ (SEQ. ID. NO. 5), were used to introduce Eco RI and Not I restriction sites. The engineered translation initiation and stop codons are underlined. For amplification of NS3/NS4A, the forward primer 5′-GTG GAA TTC ATG GCG CCT ATC ACG GCC TAT -3′ (SEQ. ID. NO. 4), and reverse primer 5′-CCC TCT AGA TCA GCA CTC TTC CAT TTC ATC-3′ (SEQ. ID. NO. 6), were used to introduce EcoRI and XbaI restriction sites. Again, the engineered translation initiation and stop codons are underlined. The expression constructs were sequenced to ensure correct sequence and reading frame and the size of the constructs was analyzed by PCR and restriction enzyme cleavage.

Expression constructs containing a mutant NS3/4A (mNS3/4A) gene were also made. In one mutant construct, for example, the amino terminal serine residue on NS4A was mutated to a proline. This mutation was introduced into the construct by site directed in vitro mutagenesis (QuikChange, Site-Directed Mutagenesis Kit, Stratagene, La Jolla, Calif.) using the forward primer (5′-CTG GAG GTC GTC ACG CCT ACC TGG GTG CTC GTT-3′ (SEQ. ID. NO. 7)) and the reverse primer (5′-AAC GAG CAC CCA GGT AGG CGT GAC GAC CTC CAG-3′ (SEQ. ID. NO. 8)). The resulting construct was the mNS3/4A-pVAX1 vector. The mutant constructs were sequenced to control the desired mutation sequence and to ensure correct reading frame.

The constructs containing NS3, the NS4A enhancer, and the mutant NS3/4A were grown and purified from E. coli cultured on LA/Kana plates containing Luria-Bertani (LB) media supplemented with 50 μg kanamycin/mL, as previously described. (Lazdina et al., J Gen Virol, 82:1299-1308 (2001) and Zhang et al., Clin Diagn Lab Immunol, 7:58-63 (2000), both of which are expressly incorporated by reference in their entireties). The purified plasmid DNA was dissolved in sterile phosphate buffer saline (PBS) to a concentration of 1 mg/ml.

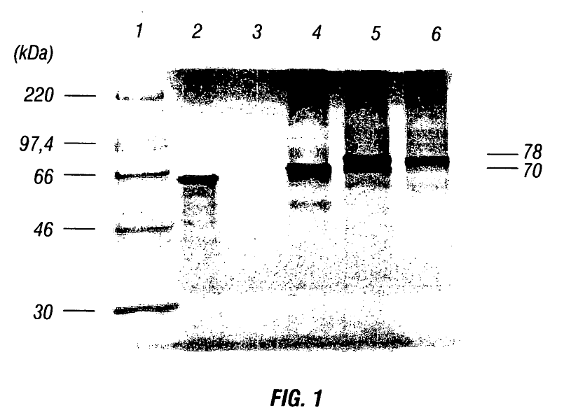

To ensure that the inserted genes were intact and could be translated, an in vitro transcription assay using the prokaryotic T7 coupled reticulocyte lysate system (TNT; Promega, Madison, Wis.) was performed in the presence of ³⁵S-methionine, as previously described. (Lazdina et al., J Gen Virol, 82:1299-1308 (2001) and Zhang et al., Clin Diagn Lab Immunol, 7:58-63 (2000)). Translation products from the plasmids NS3-pVAX1, NS3/4A-pVAX1, and mNS3/4A-pVAX1 were generated and resolved by SDS-PAGE. The assay showed that the wildtype and mutant proteins could be correctly translated from the plasmids (See FIG. 1).

It was previously observed that two bands (approx. 70 to 78 kD) become visible after in vitro translation of the NS3/4A plasmid, which indicates that the cleavage between NS3 and NS4A mediated by the NS3 protease may not be complete in the in vitro translation assay. (See Lazdina et al., J Gen Virol, 82:1299-1308 (2001) and Zhang et al., Clin Diagn Lab Immunol, 7:58-63 (2000)). By introducing a targeted mutation that replaces the P1′ serine with a proline at the NS3/4A proteolytic site (see Ingallinella et al., Biochemistry, 37:8906-8914 (1998) and Steinkuhler et al., J Virol, 70:6694-6700 (1996), herein expressly incorporated by reference in their entirities), only the band representing the expected NS3/4A fusion protein remained visible (FIG. 1). By replacing the junctional Thr-Ser-Thr motif with a Thr-Pro-Thr motif, the proteolytic site was successfully destroyed since only the NS3/4A fusion protein could be detected as a translation product from the mutant plasmid. Thus, it was determined that the NS3-pVAX1, NS3/4A-pVAX1, and mNS3/4A-pVAX1 constructs expressed the predicted full-length genes and the protease activity of NS3 remained intact.

The NS3, NS3/4A, and mNS3/4A genes were also analyzed in a Semliki forest virus (SFV) vector based expression system using Baby Hamster Kidney (BHK)-21 cells. The sequence encoding NS3, NS3/4A and mNS3/4A was isolated by PCR as Spel-BStBl fragments and inserted into the Spel-BstBl site of pSFV10Enh containing a 34 amino acid long translational enhancer sequence of capsid followed by the FMDV 2a cleavage peptide. (See Smerdou et al., Curr Opin Mol Ther, 1:244-251 (1999) and Smerdou et al., J Virol, 73:1092-1098 (1999), both of which are herein expressly incorporated by reference in their entirities).

Packaging of recombinant RNA into rSFV particles was accomplished using a two-helper RNA system. (Smerdou et al., Curr Opin Mol Ther, 1:244-251 (1999) and Smerdou et al., J Virol, 73:1092-1098 (1999)). In brief, BHK cells (maintained in complete BHK medium supplemented with 5% FCS, 10% tryptose phosphate broth, 2 mM glutamine, 20 mM Hepes and antibiotics (streptomycin 10 μg/ml and penicillin 100 IU/ml)) were co-transfected with recombinant RNA and two helper RNAs, one of which codes for the SFV capsid protein, the other for the envelope proteins. After a 48 hour incubation, medium containing recombinant virus stock was harvested and purified. (See Fleeton et al., J Gen Virol, 81:749-758 (2000), herein expressly incorporated by reference in its entirety).

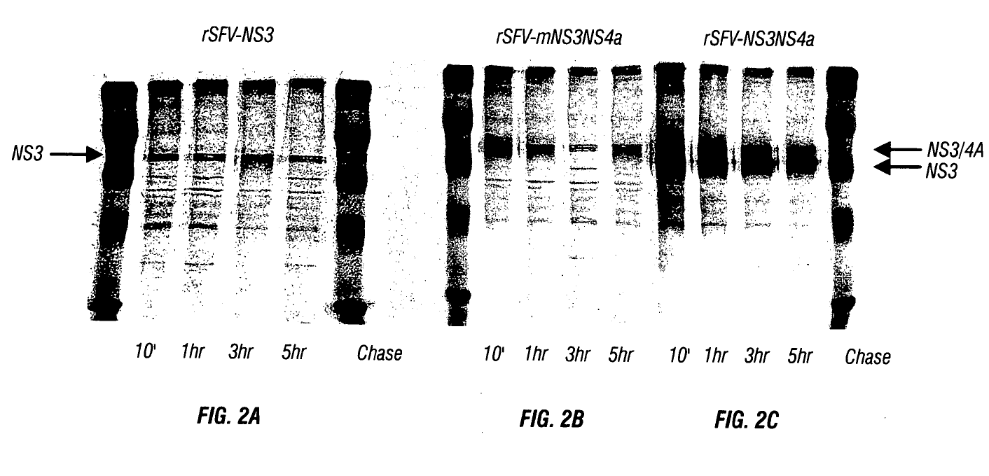

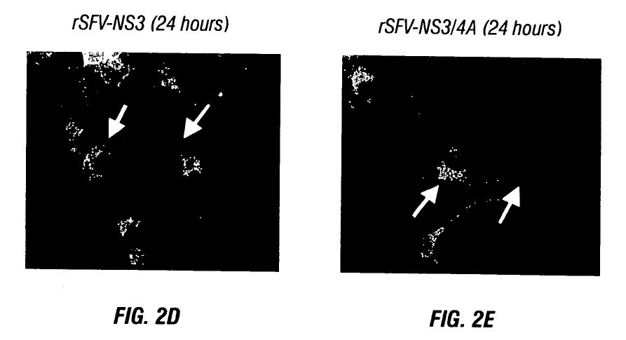

Expression levels of the rSFV infected cells were then analyzed by metabolic labeling with [³⁵S] methionine. (See Smerdou et al., Curr Opin Mol Ther, 1:244-251 (1999); Smerdou et al., J Virol, 73:1092-1098 (1999)). Briefly, BHK cells were infected with rSFV particles at a MOI of 5 and after 15 hours, the growth medium was replaced with methionine free MEM for 30 minutes prior to the addition of fresh medium containing 75 μCi/ml [³⁵S] methionine. After a 15 minute labeling period, the cells were incubated further for various times in a medium containing unlabeled methionine. Supernatants were then collected and the cells were lysed with Nonidet P-40 buffer containing 100 mM iodoacetamide. Cell lysates were immunoprecipitated with protein A sepharose and anti-NS3 monoclonal antibody (kindly provided by G. Inschauspé, Lyon, France) overnight at 4° C. The washed pellets were resuspended in SDS sample buffer, heated at 95° C. for 5 min prior to SDS-PAGE analysis on 10% acrylamide reducing gel. Nonproductive infection of BHK cells with SFV vectors expressing the three genes revealed that the NS3/4A gene with an intact proteolytic site gave the highest expression of the associated gene, NS3 (See FIG. 2). The data demonstrated that the presence of NS4A enhanced the expression of the associated gene NS3.

Indirect immunofluorescence of infected BHK cells was then performed. (Smerdou et al., Curr Opin Mol Ther, 1:244-251 (1999) and Smerdou et al., J Virol, 73:1092-1098 (1999)). Accordingly, BHK cells were infected with rSFV-NS3, NS3/4a or mNS3/4A at a MOI of 5. After 16, 18 or 24 hours of growth, the cells were fixed in methanol and protein expression was detected by incubation of the cells with anti NS3 monoclonal antibody and subsequently anti mouse IgG FITC (Sigma). Immunofluorescent staining of rSFV-NS3 and rNS3/4A infected BHK cells revealed a different intracellular distribution of NS3 (See FIG. 2). The NS3 protein expressed by infection with rSFV-NS3 displayed a more diffuse staining pattern as compared to rNS3/4A at 24 hours post infection providing evidence of the membrane targeting conferred by NS4A.

The next section describes several genes that can be associated to the NS4A enhancer in a genetic construct.

Nucleic Acids that Can be Associated with the NS4A Enhancer

The NS4A enhancer can improve the level of transcription and immunogenicity of many different associated nucleic acids. The NS4A enhancer can improve the level of transcription of a marker gene, for example. Genes encoding GFP, neomycin phosphotransferase, luciferase, lac Z or chloramphenicol transferase, among others, can be readily associated with the NS4A enhancer using commercially available constructs and/or conventional techniques in molecular biology. The NS4A enhancer can improve the level of transcription and immunogenicity of a nucleic acid encoding an immunogen, as well. Nucleic acids encoding hepatitis or HIV antigens such as peptides consisting of, consisting essentially of, or comprising peptides that correspond to sequences present on the hepatitis B virus (HBV) core and e proteins or HIV gp 120, for example, can be readily associated with the NS4A enhancer. (See e.g., U.S. Pat. Nos. 6,417,324; 5,589,175; and 5,840,313; all of which are hereby expressly incorporated by reference in their entirety). The NS4A enhancer can also improve the level of transcription of a therapeutic gene or nucleic acid fragment. Genes encoding an interferon or an interfering nucleic acid (e.g., an antisense or an RNAi generating nucleic acid) or a gene encoding an enzyme can be joined to NS4A. (See e.g., U.S. Pat. Nos. 4,855,238; 5,574,137; 5,595,888; 5,690,925; 6,326,193; or U.S. application Nos. 20020137210 and 20020086356; or PCT application Nos. WO0244321; WO0175164; WO0142443; WO0129058; WO02072762; and WO0168836, all of which are hereby expressly incorporated by reference in their entireties).

The next example describes the construction of a NS4A/GFP construct.

### EXAMPLE 2

An NS4A/GFP construct can be made and characterized as follows. A GFP vector (e.g., pDS1-1, pDS1-N1, or pDS1-C1) is obtained from a commercial supplier (Clonetech). These expression vectors are designed to evaluate the efficacy of an enhancer and/or a promoter and have convenient multiple cloning sites that facilitate the introduction of NS4A and other elements. Some vectors have endogenous promoters, for example, and others allow for a promoter to be inserted. The NS4A sequence can be generated by PCR, as described above, using primers that facilitate cloning proximal to the GFP sequence in the vector. Optionally, the promoter present in pVAX-1 is subcloned into the GFP/NS4A construct. Preferably, a control vector lacking NS4A is created so as to directly evaluate the influence of NS4A on expression of GFP. Once the correct clones are verified by sequencing, cells from a suitable cell line are transfected the NS4A/GFP construct or alternatively with the control construct. The expression of GFP in the NS4A/GFP construct containing cells and the control construct containing cells is then compared using conventional analysis (e.g, microscopy or FACS) according to the manufacturer's recommended protocols. The NS4A/GFP containing cells will show an enhanced expression of GFP as compared to cells containing the control construct.

The section below describes several ways that the NS4A enhancer was used to facilitate or improve an immune response to an associated gene.

NS4A Improves the Immunogenicity of an Associated Nucleic Acid

In addition to enhancing the level of transcription of an associated nucleic acid, it was discovered that NS4A enhanced the immunogenicity of the associated nucleic acid. Accordingly, several embodiments described herein concern the manufacture and use of constructs containing NS4A and an associated nucleic acid, which is an immunogen. The use of nucleic acids as immunogens or active ingredients in vaccine preparations is well established. (See e.g., U.S. Pat. Nos. 5,589,466 and 6,214,804, hereby expressly incorporated by reference in their entireties). Preferred embodiments concern the use of NS4A containing constructs that are associated with viral nucleic acid-based immunogens such as hepatitis immunogens (e.g., HBV core and e immunogens and HCV immunogens) and HIV immunogens (e.g., gp120 immunogens). Nucleic acids that can be associated with NS4A for this purpose include the nucleic acids and nucleic acids that encode the peptides described in U.S. Pat. Nos. 6,417,324; 5,589,175; and 5,840,313, for example. The next example describes experiments that were conducted with an NS4A containing construct, which also contained an associated NS3 gene. The results of these experiments provided evidence that NS4A facilitated or improved an immune response to an associated immunogen.

### EXAMPLE 3

To test the immunogenicity of different NS3 genes, BALB/c (H-2^{d}) mice were immunized with recombinant (r)NS3, and the NS3, NS3/4A and mNS3/4A genes and antibody titres were evaluated. BALB/c mice were used because they have been shown to be good responders to NS3 but low/non-responders to NS4A of genotype 1. (Lazdina et al., J Gen Virol, 82:1299-1308 (2001); Sallberg et al., J Gen Virol, 77:2721-2728 (1996); and Zhang et al., J Gen Virol, 78:2735-2746 (1997), all of which are hereby expressly incorporated by reference in their entireties). Thus, any differences in the immune response could not be attributed to the addition of new CD4+ T helper (Th) epitopes. The inbred BALB/c (H-2^{d}) mice were obtained from commercial vendors (Charles River, Uppsala, Sweden). Serum for antibody detection and isotyping was collected every second or fourth week after the first immunization by retroorbital bleeding of isofluorane-anesthetized mice. Enzyme immunoassays were performed as previously described. (Lazdina et al., J Gen Virol, 82:1299-1308 (2001) and Sallberg et al., J Gen Virol, 77:2721-2728 (1996)).

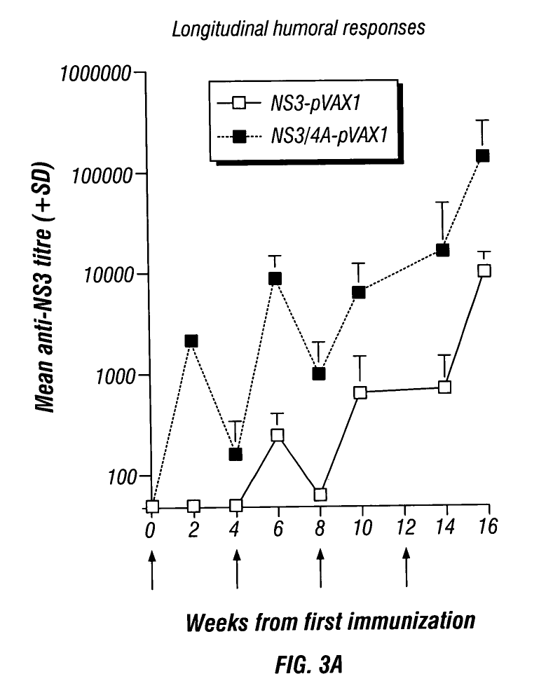

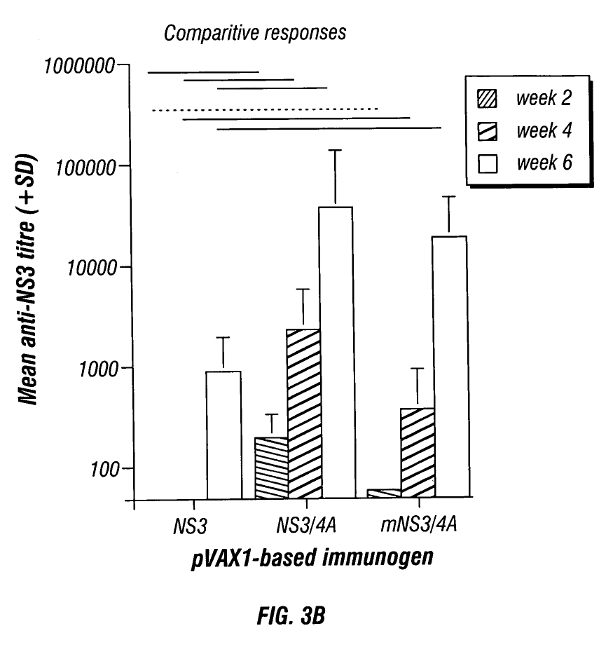

To directly compare the immunogenicity of NS3 and NS3/4A genes, two groups of five BALB/c (H-2^{d}) mice each were immunized with 100 μg NS3-pVAX1 or NS3/4A-pVAX1. Plasmid DNA in PBS was given intramuscularly (i.m.) in the tibialis anterior (TA) muscle. (Davis et al., Human Gene Therapy, 4:733-740 (1993), hereby expressly incorporated by reference in its entirety). The mice immunized with NS3/4A-pVAX1 had a more rapid antibody response, providing evidence that the NS3/4A plasmid had a higher intrinsic immunogenicity (See FIG. 3). After four immunizations, the mice that were immunized with NS3/4A had higher antibody levels.

To verify these preliminary findings, larger groups of mice were immunized with NS3-pVAX1, which only expresses NS3, or NS3/4A-pVAX1, which expresses both NS3 and NS4A, or the mutant NS3/4A plasmid, which expresses the mutant NS3/4A fusion protein. The differences in immunogenicity between NS3-pVAX1 and NS3/4A-pVAX1 plasmids were perfectly repeated. (See FIG. 3). Again, the NS3/4A gene was more immunogenic than the NS3 gene alone with respect to mean antibody levels and the frequency of responding mice. These results confirmed that NS4A enhances the immunogenicity of an associated gene and/or gene product. Interestingly, in the early immune response, i.e. at two and four weeks, the NS3/4A-pVAX1 plasmid was also more immunogenic than the mNS3/4A-pVAX1 plasmid. (See FIG. 3). Thus, in some circumstances, a functional proteolytic site between the associated gene and NS4A may be desirable.

To determine whether a new Th epitope was generated at the junction of the NS3 and NS4A proteins, which may partially explain the increased immunogenicity seen with the NS3/4A gene, T cell proliferation assays were performed. BALB/c mice were immunized with rNS3 or NS3/4A-pVAX1 and, after nine days, spleen cell recall cultures were established (i.e., in vivo primed cells were recalled for five days with rNS3 and a 20 amino acid peptide spanning the NS3/4A-junction). The recombinant NS3 (rNS3) protein was kindly provided by Darrell L. Peterson, Department of Biochemistry, Commonwealth University, VA. The production of recombinant NS3 protein (not including NS4A) in E. Coli has been described in detail previously. (Jin et al., Arch. Biochem. Biophys., 323:47-53 (1995), hereby expressly incorporated by reference in its entirety). Prior to use, the rNS3 protein was dialyzed overnight against PBS and sterile filtered. Peptide immunizations were performed using 100 μg peptide mixed with complete Freunds adjuvant (1:1), and injected subcutaneous (s.c.) in the base of the tail. The twenty-mer peptide, corresponding to the complete NS3/4A sequence used as the DNA immunogen, was synthesized by automated peptide synthesis, as previously described. (Sallberg et al., Immunol Lett, 30:59-68 (1991), herein expressly incorporated by reference in its entirety).

As shown in FIG. 3, both rNS3 and NS3/4A-pVAX1 primes T cells that were recalled in vitro by rNS3. Neither rNS3 or NS3/4A-pVAX1 primed T cells could be recalled by the NS3/4A junctional peptide. The same results were repeated in C57BL/6 (H-2^{b}) mice. These results confirmed that a new T helper cell site had not been generated by the NS3/4A fusion protein.

To compare the proliferative Th-cell responses of NS3 and NS3/4A, groups of mice were immunized with 100 μg of plasmid and, 13 days later, spleen cells were harvested and in vitro recall assays were established using rNS3. The detection of proliferative responses to NS3 followed previously described protocols. (Lazdina et al., J Gen Virol, 82:1299-1308 (2001) and Sallberg et al., J Gen Virol, 77:2721-2728 (1996)). In brief, groups of mice were immunized with 100 μg NS3-pVAX1 or NS3/4A-pVAX1 in TA muscles. Thirteen days later splenocytes were harvested, single cell suspensions were prepared and the cells were incubated with serial dilutions of rNS3. The cells were incubated with or without rNS3 for four days and for the last 24 hours ³H-labelled thymidine (TdR) was added. The uptake of radioactive thymidine was measured by liquid scintillation counting.

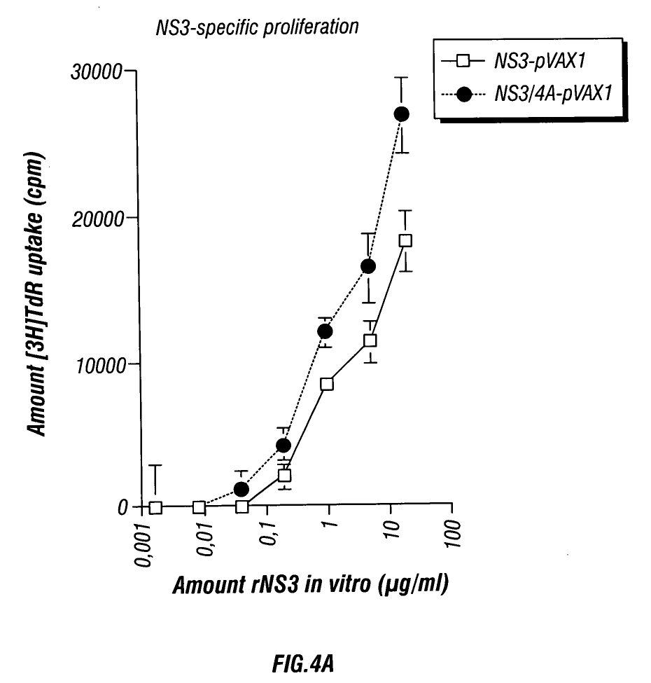

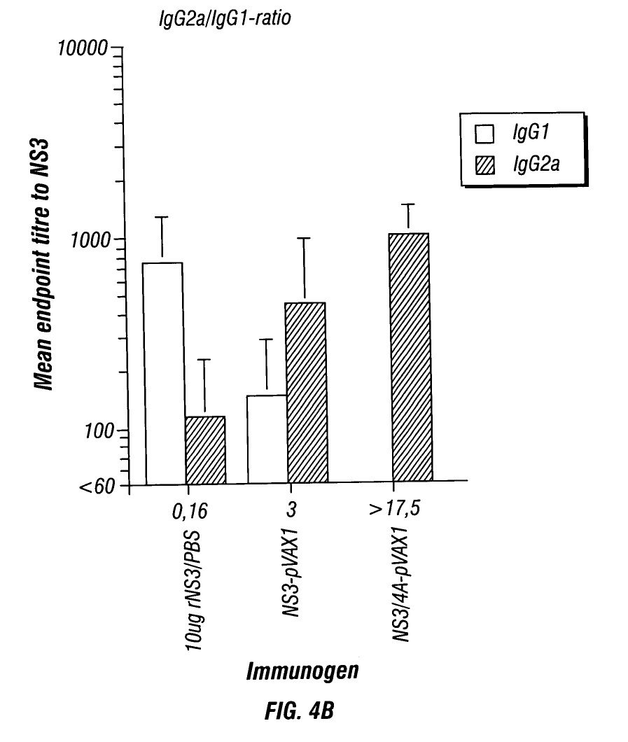

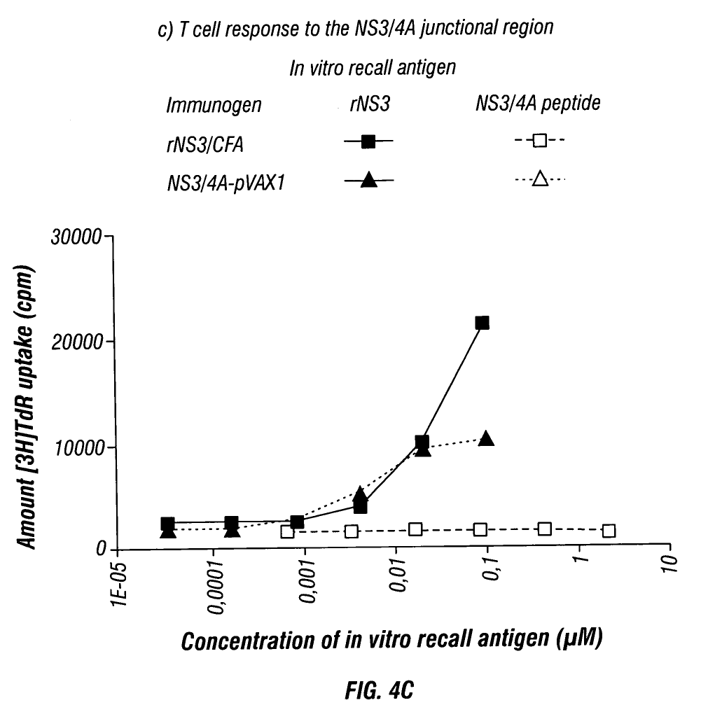

It was determined that the level of NS3-specific Th-cell priming was more efficient in the NS3/4A immunized mice than in the NS3 immunized mice (FIG. 4). The level of T cell proliferation was higher and the amount of rNS3 required to recall a detectable response was lower.

The Th-cell phenotype primed by NS3/4A immunization has been described in detail previously. (Lazdina et al., J Gen Virol, 82:1299-1308 (2001)). To directly compare the T helper 1 (Th1) and Th2-skewing of the T cell response primed by NS3 and NS3/4A immunization, the levels of NS3-specific IgG1 (Th2) and IgG2a (Th1) antibodies were analyzed. (See FIG. 4). In H-2^{d} and H-2^{k} mice immunized with rNS3 in PBS or adjuvant, IgG1 was the dominant subclass. The IgG2a/IgG1-ratio in mice immunized with rNS3 was always <1 regardless of the murine haplotype, which signals a Th2-dominated response. (Schirmbeck et al., Intervirology, 44:115-123 (2001)). In contrast, mice immunized with NS3-pVAX1 or NS3/4A-pVAX1 had Th1-skewed Th-cell responses evidenced by IgG1/IgG2a ratios of >1. However, the subclass ratio in NS3-pVAX1 immunized mice provided evidence of a mixed Th1/Th2 response (FIG. 4). In contrast, none of the NS3/4A-pVAX1 immunized mice exhibited IgG1, indicating a profoundly Th1-skewed response.

The experiments in this example demonstrated that the inclusion of NS4A in the NS3-based DNA immunogen provided for a more rapid NS3-specific humoral response, which reached higher titers. In addition, the priming of Th-cells was more effective and the Th1/Th2-balance was shifted towards Th1. Accordingly, the intrinsic immunogenicity of the NS3 protein had been improved by the addition of NS4A. The next example provides additional evidence that NS4A enhances the immunogenicity of an associated nucleic acid in vivo.

### EXAMPLE 4

An immune response to a particular antigen can be efficiently analyzed in vivo by monitoring the inhibition of tumor growth in BALB/c mice containing SP2/0 myeloma cells that express the desired antigen. (See Encke et al., J Immunol, 161:4917-4923 (1998), herein expressly incorporated by reference in its entirety). The inhibition of tumor growth following DNA immunization is fully dependent on an efficient priming of specific CTLs. (Encke et al., J Immunol, 161:4917-4923 (1998)). This model is more reliable than a recombinant vaccinia virus system, for example, because undesired viral proteins (i.e. vector derived proteins) are not produced by the cell.

An SP2/0 cell line that stably expressed NS3/4A was made and the in vivo growth kinetics of the NS3/4A-expressing cell line was found to be fully consistent with the parental cell line. The SP2/0-Ag14 myeloma cell line (H-2^{d}) was maintained in DMEM medium supplemented with 10% fetal calf serum (FCS; Sigma Chemicals, St. Louis, Mo.), 2 mM L-Glutamine, 10 mM HEPES, 100 U/ml Penicillin and 100 μg/ml Streptomycin, 1 mM non-essential amino acids, 50 μM β-mercaptoethanol, and 1 mM sodium pyruvate (GIBCO-BRL, Gaithesburgh, Md.). SP2/0-Ag14 cells having stable expression of NS3/4A were generated by transfection of SP2/0 cells with the linearized NS3/4A-pcDNA3.1 plasmid using the SuperFect (Qiagen GmbH, Hilden, FRG) transfection reagent. The transfection procedure was performed according to manufacturer's protocol. Transfected cells were cloned by limiting dilution and selected by addition of 800 μg geneticin (G418)/ml complete DMEM medium. Expression of NS3/4A was confirmed by reversed transcription PCR and by a capture EIA using a monoclonal antibody to NS3. (Zhang et al., Clin Diagn Lab Immunol, 7:58-63 (2000)).

Initial experiments were designed to determine the quantity of DNA injections that were needed to prime CTLs, which lysed the NS3/4A expressing cells in vitro. Mice were pretreated with cardiotoxin (i.m. with 50 μL/TA of 0.01 mM cardiotoxin (Latoxan, Rosans, France) in 0.9% sterile saline NaCl, five days prior to DNA immunization and were boosted at four-week intervals) and then were given two, three, or six monthly injections of 100 μg NS3/4A-pVAX1 in TA muscles. Groups of five mice were sacrificed two weeks after each injection and analyzed. Spleen cells from DNA immunized BALB/c mice were resuspended in complete DMEM medium. In vitro stimulation was carried out for five days in 25-ml flasks at a final volume of 12 ml, containing 5 U/ml recombinant murine IL-2 (mIL-2; R&D Systems, Minneapolis, Minn.). The restimulation culture contained a total of 40×10⁶ immune spleen cells and 2×10⁶ irradiated (10 000 rad) syngenic SP2/0 cells expressing the NS3/4A protein. After five days in vitro stimulation a standard ⁵¹Cr-release assay was performed. SP2/0 cells and SP2/0 cells expressing the NS3/4A protein served as targets and were labeled for one hour with 20 μl of ⁵¹Cr (5 mCi/ml) and then washed three times in PBS. Serial dilutions of effector cells were incubated with 5×10^{3 51}Cr-labeled target cells/well. After a four hour incubation at 5% CO₂, 37° C., 100 μl of supernatant was collected and the radioactivity was determined by a γ-counter.

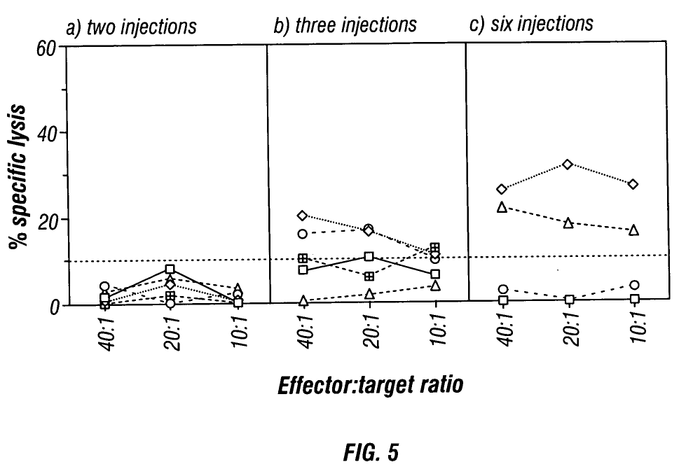

Similarly, spleen cells from peptide immunized mice (12 days post immunization) or naive mice were resuspended in RPMI 1640 medium supplemented with 10% FCS, 2 mM L-Glutamine, 10 mM HEPES, 100 U/ml Penicillin and 100 μg/ml Streptomycin, 1 mM non-essential amino acids, 50 μM β-mercaptoethanol, and 1 mM sodium pyruvate. In vitro stimulation was carried out for five days in 25-ml flasks in a total volume of 12 ml, containing 25×10⁶ spleen cells and 25×10⁶ irradiated (2000 rad) syngeneic splenocytes. The restimulation was performed in the presence of 0.05 μM NS3/4A H-2D^{b} binding peptide (sequence GAVQNEVTL SEQ. ID. No. 1) or irrelevant H-2D^{b} peptide (sequence KAVYNFATM SEQ. ID. NO. 9). After five days in vitro culture, a ⁵¹Cr-release assay was performed as described above. RMA-S cells and RMA-S cells pulsed with 50 μM peptide for 1.5 hrs at +37° C. prior to ⁵¹Cr-labelling served as targets. It was determined that three to six i.m. injections were needed to prime detectable CTLs in vitro. (See FIG. 5).

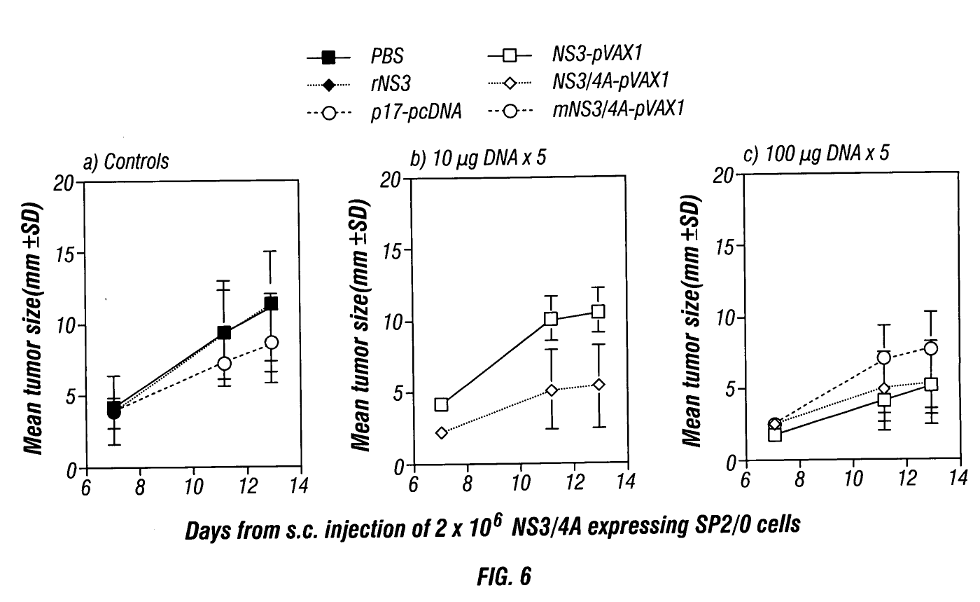

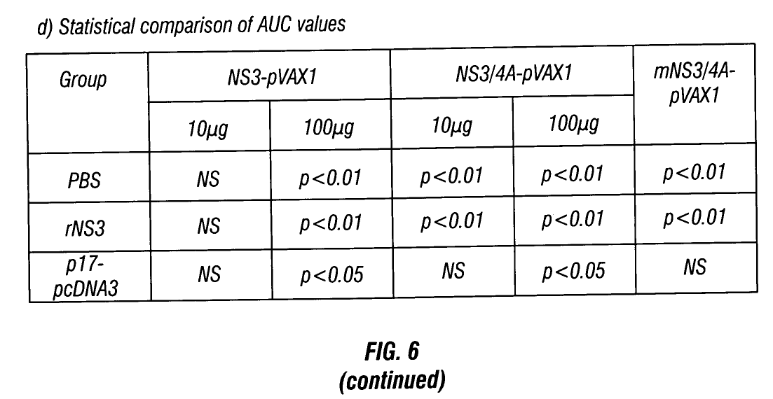

To ensure that active CTLs were primed in vivo, all mice received five immunizations prior to in vivo challenge with the NS3/4A expressing cells. In vivo challenge of immunized mice with the NS3/4A-expressing SP2/0 myeloma was performed according to the method described by Encke et al. (Encke et al., J Immunol, 161:4917-4923 (1998)). In brief, groups of BALB/c mice were immunized with different immunogens at weeks zero, four, eight, 12, and 16, as described. Two weeks after the last immunization, 2×10⁶ NS3/4A-expressing SP2/0 cells were injected s.c in the right flank. The kinetics of the tumor growth was determined by measuring the tumor size through the skin at days seven, 11, and 13. The mean tumor sizes were calculated and the kinetic tumor development were compared using the area under the curve (AUC). AUC values were compared using analysis of variance (ANOVA). Fisher's exact test was used for frequency analysis and Mann-Whitney U-test was used for comparing values from two groups. The calculations were performed using the Macintosh version of the StatView software (version 5.0). There was no difference in tumor growth among groups of mice immunized with PBS or with a control plasmid expressing the p17 protein of human immunodeficiency virus type 1 (Iroegbu et al., Clin Diagn Lab Immunol, 7:377-383 (2000), herein expressly incorporated by reference in its entirety). (See FIG. 6).

At day 14, all mice were sacrificed, the tumors were removed, paraffin embedded, and sectioned. Briefly, tumor tissue was placed in formalin, embedded in paraffin, and 4 μm sections were prepared. Paraffin-embedded sections were pre-treated with an avidin-biotin blocking kit (Vector, Vector Laboratories, Burlingame, Calif.) and then immunostained with an anti-CD3 antibody (Dako, Denmark) to determine the amount of T cell infiltration in the tumor. For detection, biotinylated immunoglobulins, followed by avidin-biotin peroxidase (Vector) were used. Microwave pre-treatment was also used for some of the CD3 immunostaining. The four μm thick tumor sections were mounted on slides and some were stained with Hematoxylin-Eosin dye, according to standard procedures. A pathologist who was blinded as to which group the section belonged, analyzed the histological appearance of the tumors.

Mice immunized with rNS3 in CFA did not show inhibition of tumor growth, confirming that the priming of specific B- and Th-cells alone does not confer tumor protection in this model, whereas mice immunized with 100 μg of NS3-pVAX1 or NS3/4A-pVAX1 showed a significant reduction in tumor growth at all time points. (See FIG. 6). Interestingly, immunization with mNS3/4A showed significant inhibition of tumor growth at days seven and 13, but not at day 11. By reducing the dose of plasmid 10-fold, the ability to prime inhibiting responses was lost for the NS3-pVAX1 plasmid, but not for the NS3/4A-pVAX1 plasmid. (See FIG. 6). These experiments revealed that NS4 enhances the immunogenicity of NS3 in the priming of tumor protecting immune responses in vivo. The presence of a functional cleavage site at the NS3/4A junction may be important because a slightly lower protection was conferred by immunization with mNS3/4A-pVAX1.

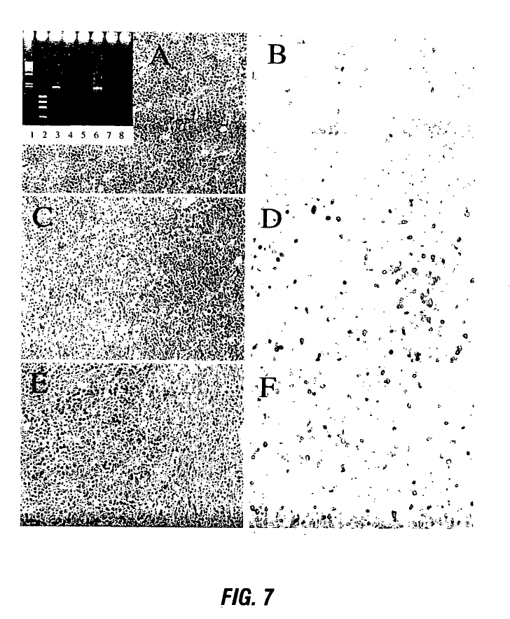

The histology of all of the harvested tumors from the different experimental groups revealed that most of the tumors that developed in the mock-immunized mice were necrotic, characterized by central cell death and the presence of pycnotic nuclear remnant. (See FIG. 7). In corresponding sections stained for CD3 antigen, only a sparse infiltration of positive T-lymphocytes was noted. Similar observations were made on tumors isolated from mice that were immunized with recombinant NS3 protein. In the DNA immunized animals (i.e., NS3-pVAX1, NS3/4A-pVAX1, and mNS3/4A-pVAX1) only occasional necrosis was observed. In these tumors, large areas had been replaced by oedematous and vascularized tissue. (See FIG. 7). These areas were densely infiltrated by CD3 positive lymphocytes. At the interface to viable tumor tissue, an accumulation of lymphocytes was noted, as well as apoptotic cells, which may be dying myeloma cells. (See FIG. 7). In addition, staining with the CD3 antibody revealed a major invasion of T cells in areas of tumor regression. (See FIG. 7).

These data further confirm that T cells, presumably CTLs, are responsible for the observed inhibition of tumor growth and that CTL-dependent inhibition of NS3/4A-expressing tumor cells could be obtained in vivo at 10-fold lower doses of the immunogen when NS4A was present. Clearly, NS4A enhances the immunogenicity of an associated gene or gene product (e.g., heterodimer or fusion protein).

The next example describes more experiments that evaluated the use of a NS3/NS4A based DNA immunogen.

### EXAMPLE 5

Although injections in regenerating muscle tissue are effective for DNA immunizations in mice, such treatments are not desirable for human use. Accordingly, experiments were conducted to evaluate the efficacy of transdermal immunization with the NS3/4A-pVAX1 immunogen using a gene gun. For gene gun based immunizations, plasmid DNA was linked to gold particles according to protocols supplied by the manufacturer (Bio-Rad Laboratories, Hercules, Calif.). Prior to immunization, the injection area was shaved and the immunization was performed according to the manufacturer's protocol. Each injection dose contained 4 μg of plasmid DNA. The mice were boosted with the same dose at monthly intervals.

Initially, the reagents needed to quantify the CTL responses by flow cytometry were developed so as to evaluate the CTL priming efficiency of transdermal plasmid administration. First, a peptide corresponding to an H-2^{b}-restricted NS3-specific CTL epitope was identified so as to quantify NS3/4A-specific CTLs using a divalent MHC:Ig fusion protein (See Dal Porto et al,. Proc Natl Acad Sci USA, 90:6671-6675 (1993), herein expressly incorporated by reference in its entirety). Next, NS3/4A-specific CTL epitopes were identified from a set of overlapping 20 amino acid long synthetic peptides spanning NS3/4A (in total 69 different peptides with 10 amino acid overlap). The 20 amino acid long peptides were assayed for stabilization of surface expression of MHC class I molecules on a transporter associated with an antigen processing (TAP) 2 deficient RMA-S cell line. (Ljunggren et al., Nature, 346:476-480 (1990); Stuber et al., Eur J Immunol, 22:2697-2703 (1992), herein expressly incorporated by reference in its entirety).

Briefly, RMA-S cells were maintained in RPMI 1640 medium supplemented with 5% FCS, 2 mM L-Glutamine, 100 U/ml Penicillin and 100 μg/ml Streptomycin. All cells were grown in a humidified 37° C., 5% CO₂ incubator. Approximately, 1×10⁶ RMA-s cells were incubated in RPMI 1640 medium supplemented with 10% FCS, 2 mM L-Glutamine and 10 mM HEPES for 16-20 hours with about 0.3 mM of individual 20-mer peptides at room temperature (˜21 ° C.). Cells were then washed and stained for 30 minutes on ice with optimal concentration (1 μg/10⁶) of FITC conjugated anti-H-2K^{b} or anti-H-2D^{b} antibodies. Cells were resuspended in PBS/1% FCS (FACS buffer) containing 0.5 μg/ml of Propidium Iodine (PI; Sigma). The H-2K^{b} and H-2D^{b} expression on live cells (PI negative) were then analyzed by FACS. By this assay, a single peptide was identified, which bound H-2D^{b} molecules with high affinity.

To identify a preferable peptide sequence, nine amino acid long peptides (an eight amino acid overlap) were synthesized and evaluated for H-₂D^{b} binding. Varying peptide concentrations (0.01-100 μM) were used and the peptide loaded RMA-S cells were chased at 37° C. for 45 minutes prior to staining with anti-H-2D^{b} antibodies in order to reduce non-specific background.

The experiments above revealed a peptide consisting of the sequence GAVQNEVTL SEQ. ID. NO. 1, located at the C-terminal domain of NS3, 21-amino acids from the NS3/4A junction that significantly bound H-2D^{b}. This peptide was then used to immunize C57BL/6 (H-2^{b}) mice (4-8 weeks old). The inbred C57BL/6 (H-2^{b}) mice were obtained from commercial vendors (Charles River, Uppsala, Sweden). Splenocytes from the immunized mice were harvested and restimulation cultures were set with the NS3 peptide and irrelevant peptides. Five days later the effector cells were tested for lysis of peptide loaded RMA-S cells.

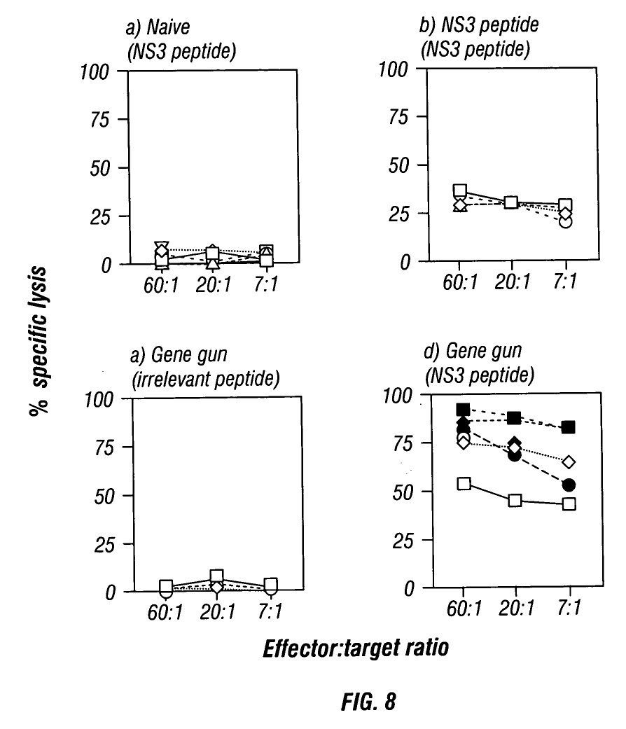

NS3/4A-specific CTLs could only be detected in splenocytes from peptide immunized mice that had been restimulated with the NS3/4A-peptide. (See FIG. 8). To test whether the NS3-derived CTL peptide could be recognized by CTLs primed by NS3/4A-pVAX1 immunization using gene gun, spleens from DNA immunized mice were restimulated with the NS3-peptide and evaluated for lysis of peptide loaded RMA-S cells. These experiments showed that mice immunized transdermally with NS3/4A-pVAX1 using a gene gun developed NS3-specific CTLs only when splenocytes had been restimulated with the NS3-peptide and not an irrelevant peptide (See FIG. 8).

The specific CTLs were then quantified directly ex-vivo. One advantage of this approach was that it circumvented in vitro expansion of CTLs prior to analysis. The frequency of NS3/4A-peptide specific CD8+ T cells were analyzed by ex-vivo staining of spleen cells from NS3/4A DNA immunized mice with recombinant soluble dimeric mouse H-₂D^{b}:Ig fusion protein. Approximately 2×10⁶ spleen cells, resuspended in 100 μl PBS/1% FCS (FACS buffer), were incubated with 1 μg/10⁶ cells of Fc-blocking antibodies on ice for 15 minutes. The cells were then incubated on ice for 1.5 hrs with either 2 μg/10⁶ cells of H-2D^{b}:Ig preloaded for 48 hours at +4° C. with 160 nM excess of NS3/4A derived peptide (sequence GAVQNEVTL SEQ ID NO. 1) or 2 μg/10⁶ cells of unloaded H-2D^{b}:g fusion protein. The cells were then washed twice in FACS buffer and resuspended in 100 μl FACS buffer containing 10 μl/100 μl PE conjugated Rat-a Mouse IgG1 secondary antibody and incubated on ice for 30 minutes. The cells were then washed twice in FACS buffer and incubated with 1 μg/10⁶ cells of FITC conjugated a-mouse CD8 antibody for 30 minutes. The cells were then washed twice in FACS buffer and resuspended in 0.5 ml FACS buffer containing 0.5 μg/ml of PI. Approximately 200,000 events from each sample were acquired on a FACS Calibur (BDB) and dead cells (PI positive cells) were excluded in the analysis.

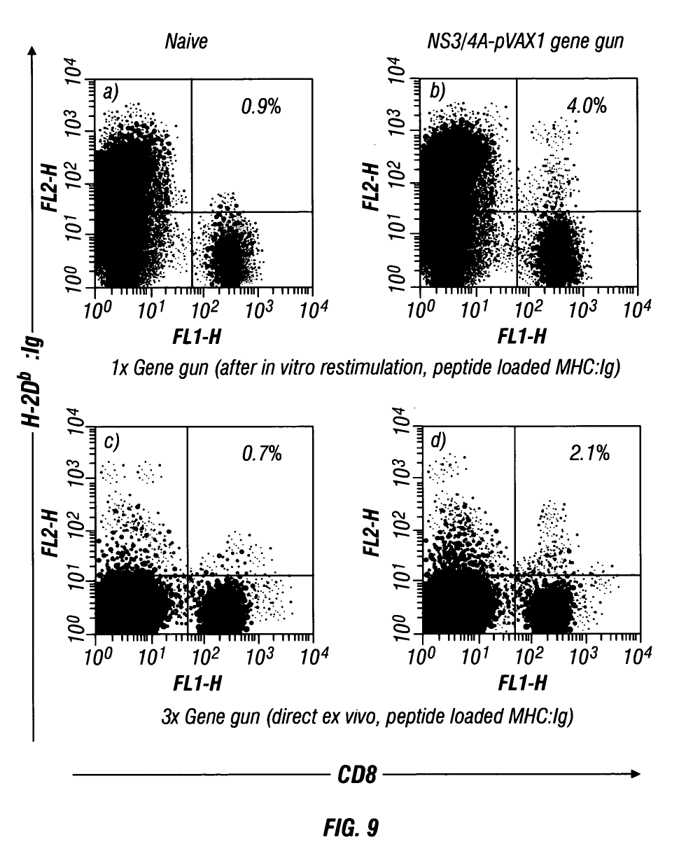

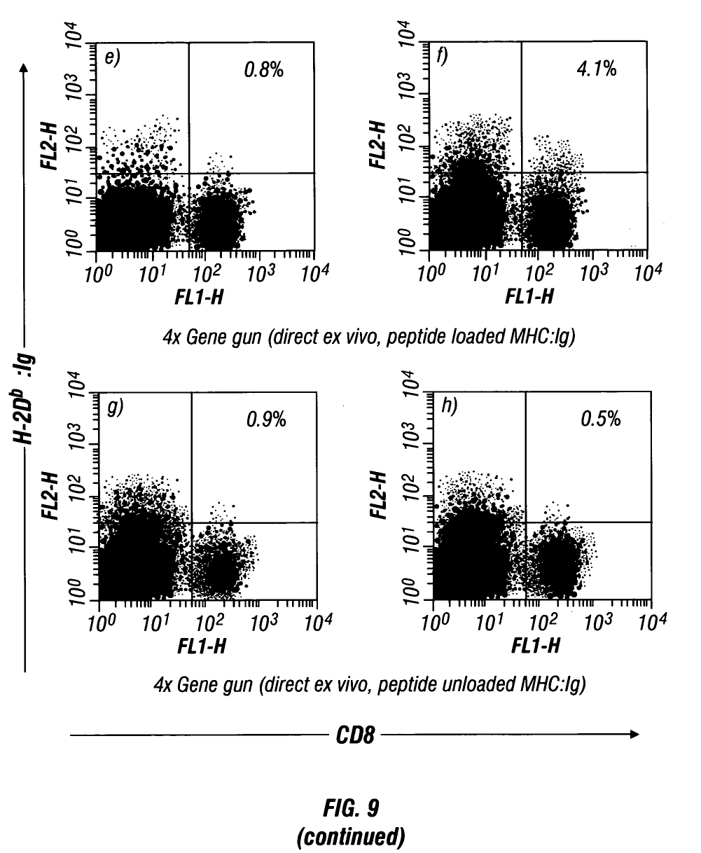

Direct ex-vivo quantification of NS3-specific CTLs using NS3-peptide loaded divalent H-2D^{b}:Ig fusion protein molecules revealed that around 2% to 4% of the CD8+ population in the spleens from mice immunized transdermally with NS3/4A-pVAX1 using the gene gun were specific for NS3/4A (See FIG. 9). This result was fully consistent with the effective lysis of peptide-loaded cells recorded in the lytic assays. Clearly, NS3/4A-pVAX1 effectively primed a large population of specific CTLs, which were readily detectable in vitro and recognized a finely mapped H-2D^{b} binding NS3-specific CTL epitope.

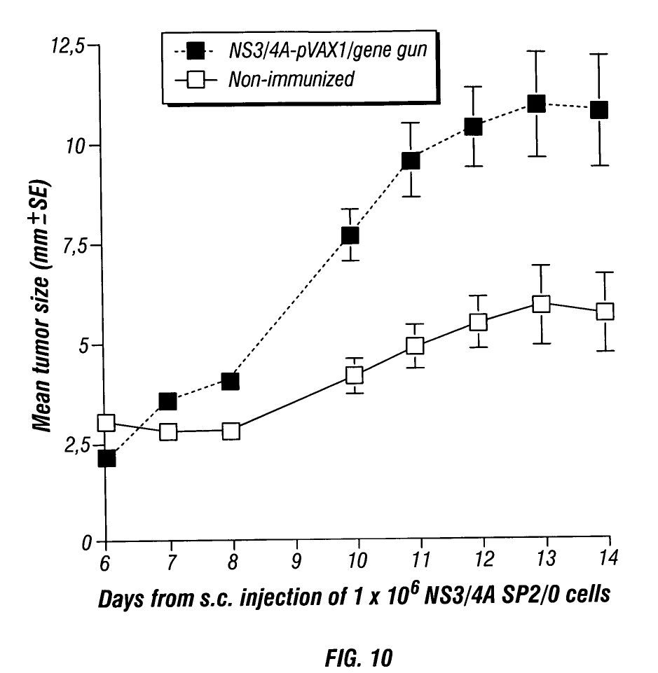

To test the efficiency of the in vivo primed NS3/4A-specific CTL responses following transdermal administration, immunized mice were challenged with the NS3/4A expressing SP2/0 tumor cell line. Previous experiments had shown that four transdermal injections primed a high precursor frequency of NS3/4A-specific CTLs. Groups of ten BALB/c mice were either left untreated or given four injections with 4 μg of the NS3/4A-pVAX1 plasmid at monthly intervals. A total dose of 16μg NS3/4A-pVAX1 plasmid effectively primed CTL responses in vivo and significantly inhibited tumor growth. (See FIG. 10). Thus, by using gene gun immunization with an antigen dose consistent with that already used in human vaccine trials (Roy et al., Vaccine, 19:764-778 (2000), herein expressly incorporated by reference in its entirety), it was discovered that the NS3/4A-pVAX1 plasmid effectively primed a tumor inhibiting immune response. The next example provides evidence that NS4A enhances the immunogenicity of an associated nucleic acid by increasing its expression.

### EXAMPLE 6

To evaluate the basis for the increased immunogenicity of genes associated with NS4A, experiments were performed to study B cell activation and proliferation in the presence of NS3/4A-pVAX1 plasmid or control sequences. BALB/c splenocytes (2×10⁶/ml) in RPMI 1640 medium, 10% FCS were stimulated for 24 hrs or 48 hrs with 5 μg/ml pVAX1 vector or 5 μg/ml NS3-pVAX1 DNA or 5 μg/ml NS3/4A-pVAX1 DNA. Cells grown in medium only served as a negative control, and 1 μg/ml LPS (Sigma Chemicals, St. Louis, Mo.) and 1.3 μg/ml of a Phosphorothioate-modified oligodeoxynucleotide (ODN; Cybergene AB, Sweden) termed CpG-1826 (Hartmann et al., J Immunol, 164:1617-1624 (2000)) served as positive controls. During the last 4 hrs of culture, bromodeoxyuridine (BrdU; Sigma Chemicals) was added to a final concentration of 10 μM. At the end of culture, cells were centrifuged and washed 2 times in PBS/1% FCS. After the final wash, cells were incubated with 2.4G2 mAb (1 μg/10⁶ cells in PBS/1% FCS) for 20 min at +4 ° C. Cells were then washed as above. Thereafter cells were stained with PE-conjugated anti-CD69 antibody and CyChrome™-conjugated anti-CD45R/B220 antibody for 30 min at +4 ° C. Cells were then washed as above. Thereafter cells were fixed and permeabilized by adding 100 μl Cytofix/Cytoperm™ solution (included in Cytofix/Cytoperm Plus kit; BDB Pharmingen) per well and incubated for 20 min at +4 ° C. Cells were thereafter washed in Perm/Wash™ solution (included in Cytofix/Cytoperm Plus kit). Cells were stained with 1:10 of FITC-conjugated anti-BrdU antibody diluted in Perm/Wash™ solution supplemented with 2.5 μl/ml of a 2000 U/ml (50 mg/ml PBS) DNase I purchased from Boehringer Mannheim (Mannheim, Germany). Cells were incubated for one hour in the dark at room temperature and then washed twice in Perm/Wash™ solution and resuspended in PBS/1% FCS. Samples were analysed on a FACS Calibur™ (BDB) and the percentage of B cells (CD45R/B220 gate) positive for CD69 and BrdU were calculated using the CellQuest™ (BDB) program. It was observed that the control DNA sequence (CPG-1826) activated B cells but a comparison of B cell activation induced by the addition of NS3-pVAX1 and NS3/4A-pVAX-1 by flow cytometry showed no difference. This data provided evidence that NS4A increases the immunogenicity of an associated gene by enhancing the expression of the associated gene.

In the preceding examples, all monoclonal antibodies and MHC:Ig fusion proteins (Dal Porto et al,. Proc Natl Acad Sci USA, 90:6671-6675 (1993)) were purchased from BDB Pharmingen (San Diego, Calif.) including: Anti-CD16/CD32 (Fc-block™, clone 2.4G2), FITC conjugated anti-CD8 (clone 53-6.7), FITC conjugated anti-H-2K^{b} (clone AF6-88.5), FITC conjugated anti-H-2D^{b} (clone KH95), recombinant soluble dimeric mouse H-2D^{b}:Ig, PE conjugated Rat-α Mouse IgG1 (clone X56), FITC conjugated anti-BrdU (clone B44), PE conjugated anti-CD69 (clone H1.2F3), Cy-Chrome™ conjugated anti-CD45R/B220 (clone RA3-682). The next example provides more evidence that NS4A is an enhancer.

### EXAMPLE 7

To directly compare the in vitro lytic activity of the NS3-specific CTLs primed by different vectors, a standard ⁵¹Cr-release assay was performed after one or two immunizations. Priming of in vitro detectable CTLs in H-2^{b} mice was conducted by gene gun immunization of the wtNS3-pVAX1 (wild-type NS3), wtNS3/4A (wild-type NS3/4A), and coNS3/4A (human codon-optimized NS3/4A) plasmids, or s.c. injection of wtNS3/4A-SFV particles (NS3/4A containing Semliki Forest virus particles). To create the codon-optimized NS3/4A construct, wild-type NS3/4A was analyzed for codon usage with respect to the most commonly used codons in human cells. A total of 433 nucleotides (15 amino acids differed) were replaced to optimize codon usage for human cells. The coNS3/4A gene has a sequence homology of 79% with the region at nucleotide positions 3417-5475 of the HCV-1 reference strain.

Groups of five to 10 H-2^{b} mice were immunized once (a) or twice (b). The lytic activity of the in vivo primed CTLs were assayed on both NS3-peptide loaded H-2D^{b} expressing RMA-S cells and EL-4 cells stably expressing NS3/4A. The percent specific lysis corresponds to the percent lysis obtained with either NS3-peptide coated RMA-S cells (upper panel in (a) and (b) or NS3/4A-expressing EL-4 cells (lower panel in (a) and (b) minus the percent lysis obtained with unloaded or non-transfected EL-4 cells. Values have been given for effector to target (E:T) cell ratios of 60:1, 20:1 and 7:1. Each line indicates an individual mouse.

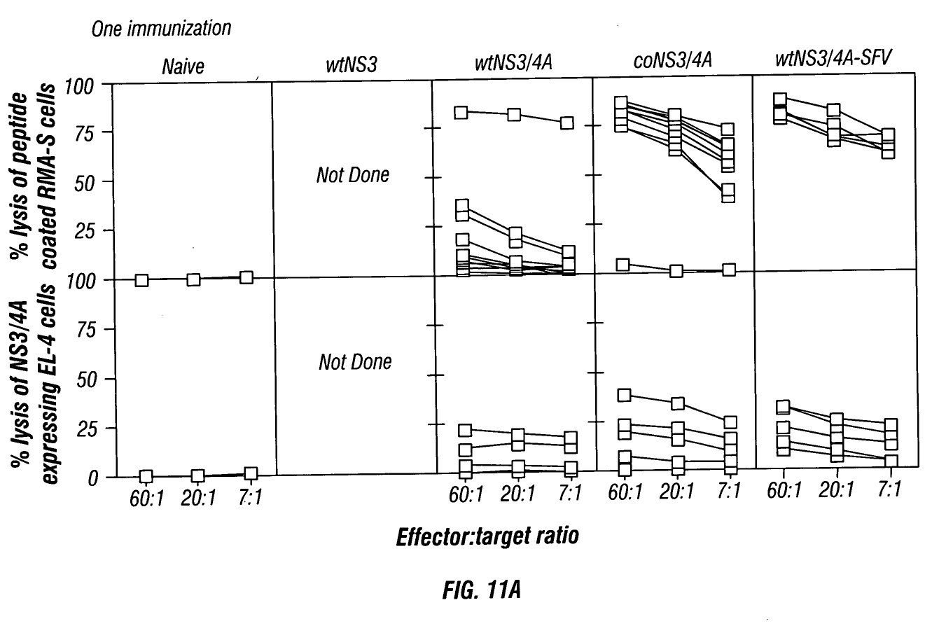

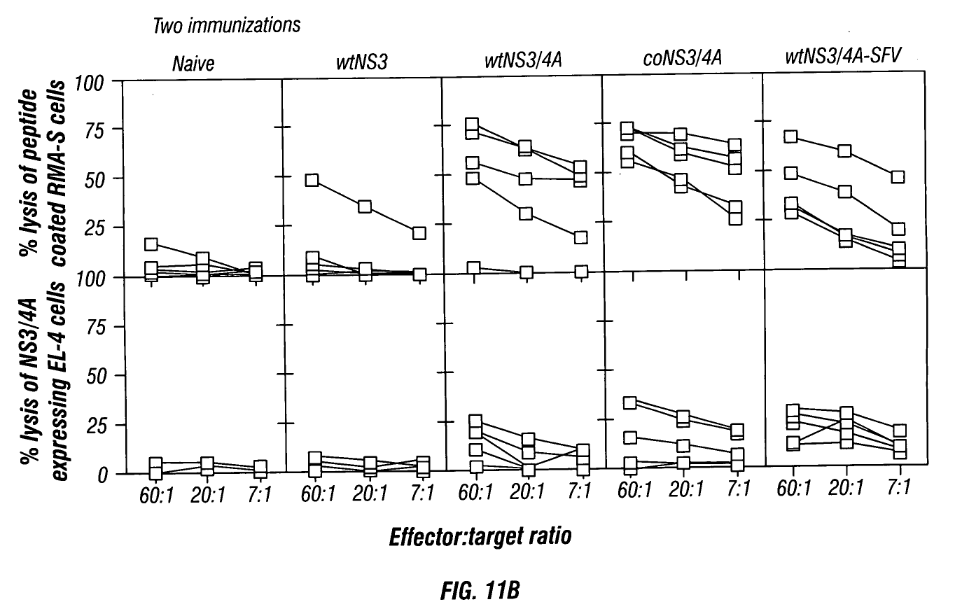

After one dose, it became apparent that the NS3/4A encoding constructs were significantly more efficient than the NS3 plasmid in priming CTLs that lysed NS3-peptide coated target cells (see FIG. 11). Thus, the CTL priming event was enhanced by the presence of the NS4A gene. The difference was less clear when using the NS3/4A-expressing EL-4 cells presumably since this assay is less sensitive. After two immunizations all NS3/4A vectors seemed to prime NS3-specific CTLs with a similar efficiency. However, two immunizations with any of the NS3/4A-containing vectors were clearly more efficient in priming NS3-specific CTLs, as compared to the plasmid containing only the wtNS3 gene. Thus, the NS4A gene is an enhancer that promotes a more rapid priming of NS3-specific CTLs. The next example provides even more evidence that NS4A is an enhancer.

### EXAMPLE 8

Analysis of the inhibition of tumor growth in vivo in BALB/c mice using SP2/0 myeloma cells, or in C57BL/6 mice using EL-4 lymphoma cells, expressing an HCV viral antigen is recognized by those in the field to represent the in vivo functional HCV-specific immune responses. (See Encke J et al., J Immunol 161: 4917-4923 (1998)). An SP2/0 cell line stably expressing NS3/4A has previously been described (see Frelin L et al., Gene Ther 10: 686-699 (2003)) and an NS3/4A expressing EL-4 cell line was characterized as described below.

To confirm that inhibition of tumor growth using the NS3/4A-expressing EL-4 cell line is fully dependent on an NS3/4A-specific immune response, a control experiment was performed. Groups of ten C57BL/6 mice were either left non-immunized, or immunized twice with the coNS3/4A plasmid. Two weeks after the last immunization the mice were challenged with a s.c. injection of 10⁶ native EL-4 or NS3/4A-expressing EL-4 cells (NS3/4A-EL-4). An NS3/4A-specific immune response was required for protection, since only the immunized mice were protected against growth of the NS3/4A-EL-4 cell line. Thus, this H-2^{b}-restricted model behaves very similar to the previously described H-2^{d} restricted model (Id.).

Immunizations with recombinant NS3 protein provided evidence that both NS3/4A-specific B cells and CD4+ T cells were not of a pivotal importance in protection against tumor growth. In vitro depletion of CD4+ or CD8+ T cells of splenocytes from coNS3/4A plasmid immunized H-2^{b} mice suggested that CD8+ T cells were the major effector cells in the ⁵¹Cr-release assay. To define the in vivo functional anti-tumor effector cell population, CD4+ or CD8+ T cells in mice immunized with the coNS3/4A plasmid were selectively depleted one week prior to, and during, challenge with the NS3/4A-EL-4 tumor cell line. Analysis by flow cytometry revealed that 85% of CD4+ and CD8+ T cells had been depleted, respectively. This experiment revealed that in vivo depletion of CD4+ T cells had no significant effect on the tumor immunity. In contrast, depletion of CD8+ T cells in vivo significantly reduced the tumor immunity. Thus, as expected, NS3/4A-specific CD8+ CTLs seems to be the major protective cell at the effector stage in the in vivo model for inhibition of tumor growth.

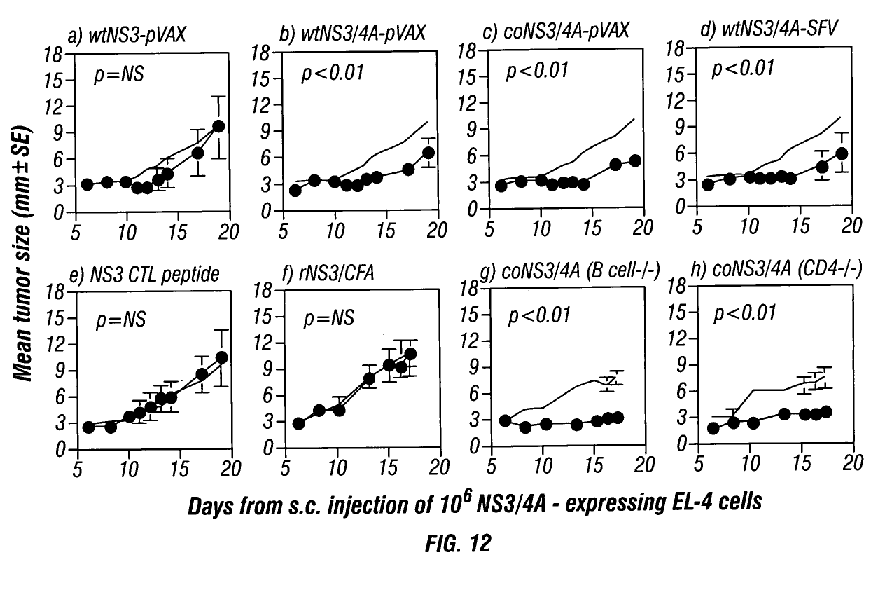

The tumor challenge model described above was then used to evaluate the efficiency of the different immunogens in priming a protective immunity against growth of NS3/4A-EL-4 tumor cells in vivo. To ensure that the effectiveness of the priming event was studied, all mice were immunized only once. Fully consistent with the in vitro CTL data, it was observed that only vectors containing NS3/4A were able to rapidly prime protective immune responses. See FIG. 12 (p<0.05, ANOVA). This priming event was dependent on the NS4A enhancer and independent of codon optimization.

To further clarify the prerequisites for priming of the in vivo protective CD8+ CTL responses, additional experiments were performed. First, C57BL/6 mice immunized with the NS3-derived CTL peptide were not protected against growth of NS3/4A-EL-4 tumors (FIG. 12). Second, immunization with recombinant NS3 in adjuvant did not protect against tumor growth (FIG. 12). Because NS3-derived CTL peptide effectively primes CTLs in C57BL/6 mice and rNS3 in adjuvant primes high levels of NS3-specific T helper cells, an endogenous production of NS3/4A appears to be needed to prime in vivo protective CTLs. To further characterize the priming event, groups of B cell (μMT) or CD4 deficient C57BL/6 mice were immunized once with the coNS3/4A gene using gene gun, and were challenged two weeks later (FIG. 12). Since both lineages were protected against tumor growth, neither B cells or CD4+ T cells were required for the priming of in vivo functional NS3/4A-specific CTLs (FIG. 12). Thus, the priming of in vivo tumor protective NS3/4A-specific CTLs in C57BL/6 mice requires the enhancer NS4A and an endogenous expression of the immunogen. In C57BL/6 mice the priming is less dependent on the gene delivery route or accessory cells, such as B cells or CD4+ T cells. The fact that the priming of in vivo functional CTL by the coNS3/4A DNA plasmid was independent of CD4+ T helper cells may help to explain the speed by which the priming occurred.

Repeated experiments in C57BL/6 mice using the NS3/4A-EL-4 cell line have shown that protection against tumor growth is obtained already after the first immunization with the NS3/4A gene, independent of codon optimization (FIG. 12). Also, after two injections the immunity against NS3/4A-EL-4 tumor growth was even further enhanced, but only when NS4A was present. Thus, this model may therefore not be sufficiently sensitive to reveal subtle differences in the intrinsic immunogenicity of different immunogens. To better compare the immunogenicity of the wtNS3/4A and the coNS3/4A DNA plasmids, additional experiments were performed in H-2^{d} mice, wherein at least two immunizations appeared to be required for a tumor protective immunity. It is important to point out that the IgG subclass distribution obtained after gene gun immunization with the NS3/4A gene in BALB/c mice showed a mixed Th1/Th2-like response. Thus, it was possible that a Th2-like immunization route (gene gun) in the Th2-prone BALB/c mouse strain may impair the ability to prime in vivo effective CTL responses.

The compositions described herein may contain other ingredients including, but not limited to, various peptides, adjuvants, binding agents, excipients such as stabilizers (to promote long term storage), emulsifiers, thickening agents, salts, preservatives, solvents, dispersion media, coatings, antibacterial and antifungal agents, isotonic and absorption delaying agents and the like. See e.g., U.S. application Ser. No. 09/929,955 and U.S. application Ser. No. 09/930,591, both of which are hereby expressly incorporated by reference in their entireties. These compositions are suitable for treatment of animals, particularly mammals, either as a preventive measure to avoid a disease or condition or as a therapeutic to treat animals already afflicted with a disease or condition.

Many other ingredients can be also be present. For example, the adjuvant and antigen can be employed in admixture with conventional excipients (e.g., pharmaceutically acceptable organic or inorganic carrier substances suitable for parenteral, enteral (e.g., oral) or topical application that do not deleteriously react with the adjuvant and/or antigen). Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable carriers are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975).

The gene constructs described herein, in particular, can be formulated with or administered in conjunction with agents that increase uptake and/or expression of the gene construct by the cells relative to uptake and/or expression of the gene construct by the cells that occurs when the identical genetic vaccine is administered in the absence of such agents. Such agents and the protocols for administering them in conjunction with gene constructs are described in PCT patent application Ser. N. PCT/US94/00899 filed Jan. 26, 1994. Examples of such agents include: CaPO₄, DEAE dextran, anionic lipids; extracellular matrix-active enzymes; saponins; lectins; estrogenic compounds and steroidal hormones; hydroxylated lower alkyls; dimethyl sulfoxide (DMSO); urea; and benzoic acid esters anilides, amidines, urethanes and the hydrochloride salts thereof, such as those of the family of local anesthetics. In addition, the gene constructs can be encapsulated within/administered in conjunction with lipids/polycationic complexes.

A nucleic acid encoding NS4A can be provided in “cis” with the gene to be enhanced (e.g., side-by-side or juxtaposed) or can be provided in “trans” (e.g., on a separate construct that operates independent of a construct containing the gene to be enhanced or on a separate construct that cointegrates with the construct containing the gene to be enhanced). Alternatively, NS4A peptide can be administered in conjunction with any of the constructs described above.

Vaccines can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the adjuvant or the administered nucleic acid or peptide.

The effective dose and method of administration of a particular vaccine formulation can vary based on the individual patient and the type and stage of the disease, as well as other factors known to those of skill in the art. Therapeutic efficacy and toxicity of the vaccines can be determined by standard pharmaceutical procedures in cell cultures or experimental animals, e.g., ED₅₀ (the dose therapeutically effective in 50% of the population). The data obtained from cell culture assays and animal studies can be used to formulate a range of dosage for human use. The dosage of the vaccines lies preferably within a range of circulating concentrations that include the ED₅₀ with no toxicity. The dosage varies within this range depending upon the type of adjuvant derivative and HCV antigen, the dosage form employed, the sensitivity of the patient, and the route of administration.

Since many adjuvants have been on the market for several years, many dosage forms and routes of administration are known. All known dosage forms and routes of administration can be provided within the context of the embodiments described herein. Preferably, an amount of adjuvant that is effective to enhance an immune response to an antigen in an animal can be considered to be an amount that is sufficient to achieve a blood serum level of antigen approximately 0.25-12.5 μg/ml in the animal, preferably, about 2.5 μg/ml. In some embodiments, the amount of adjuvant is determined according to the body weight of the animal to be given the vaccine. Accordingly, the amount of adjuvant in a vaccine formulation can be from about 0.1-6.0 mg/kg body weight. That is, some embodiments have an amount of adjuvant that corresponds to approximately 0.1-1.0 mg/kg, 1.1-2.0 mg/kg, 2.1-3.0 mg/kg, 3.1-4.0 mg/kg, 4.1-5.0 mg/kg, and 5.1-6.0 mg/kg body weight of an animal. More conventionally, the vaccines contain approximately 0.25 mg -2000 mg of adjuvant. That is, some embodiments have approximately 250 μg, 500 μg, 1 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, and 2 g of adjuvant.

As one of skill in the art will appreciate, the amount of antigens in a vaccine can vary depending on the type of antigen and its immunogenicity. The amount of antigens in the vaccine can vary accordingly. Nevertheless, as a general guide, the vaccines can have approximately 1 μg, 5 μg, 1 μg, 20 μg, 40 μg, 80 μg, 100 μg, 0.25 mg-5 mg, 5-10 mg, 10-100 mg 100-500 mg, and upwards of 2000 mg of an antigen described herein, for example. Preferably, the amount of antigen is 0.1 μg-1 mg, desirably, 0.1 μg-100 μg, preferably 3 μg-50 μg, and, most preferably, 7 μg, 8 μg, 9 μg, 10 μg, 11 μg-20 μg, when said antigen is a nucleic acid.

In some approaches described herein, the exact amount of adjuvant and/or antigen is chosen by the individual physician in view of the patient to be treated. Further, the amounts of adjuvant can be added in combination to or separately from the same or equivalent amount of antigen and these amounts can be adjusted during a particular vaccination protocol so as to provide sufficient levels in light of patient-specific or antigen-specific considerations. In this vein, patient-specific and antigen-specific factors that can be taken into account include, but are not limited to, the severity of the disease state of the patient, age, and weight of the patient, diet, time and frequency of administration, drug combination(s), reaction sensitivities, and tolerance/response to therapy.

Although the invention has been described with reference to embodiments and examples, it should be understood that various modifications can be made without departing from the spirit of the invention. Accordingly, the invention is limited only by the following claims. All references cited herein are expressly incorporated by reference in their entireties.

1. A method of increasing the expression of a nucleic acid in a cell comprising:
providing a first nucleic acid encoding an hepatitis C virus (HCV) non-structural protein 4A (NS4A) or a functional portion thereof;
identifying a second nucleic acid for increased expression;
associating said second nucleic acid with said first nucleic acid in said cell, whereby such association results in an expression of said second nucleic acid that is greater than in the absence of said first nucleic acid.

2. The method of claim 1, wherein said second nucleic acid is HCV non-structural protein 3 (NS3).

3. The method of claim 1 wherein said first and second nucleic acid are joined in cis.

4. The method of claim 1 wherein said first and second nucleic acid are juxtaposed.

5. The method of claim 1 wherein said first and second nucleic acid are on the same construct.

6. The method of claim 1, wherein said first and second nucleic acid are on separate constructs.

7. The method of claim 1, wherein said first nucleic acid consists of between 10 and 20 consecutive amino acids of SEQ. ID. NO. 2.

8. The method of claim 1, wherein said first nucleic acid consists of between 20 and 30 consecutive amino acids of SEQ. ID. NO. 2.

9. The method of claim 1, wherein said first nucleic acid consists of between 30 and 40 consecutive amino acids of SEQ. ID. NO. 2.

10. The method of claim 1, wherein said first nucleic acid consists of between 50 and 54 consecutive amino acids of SEQ. ID. NO. 2.

11. A method of increasing immunogenicity to an antigen in a mammal comprising:
providing a first nucleic acid encoding an hepatitis C virus (HCV) non-structural protein 4A (NS4A) or a functional portion thereof;
identifying a second nucleic acid that encodes an antigen for which an increased immunogenicity in a mammal is needed;
associating said second nucleic acid with said first nucleic acid, whereby such association generates an immunogenicity to said antigen in said mammal that is greater than the immunogenicity to said antigen generated by said second nucleic acid in the absence of said first nucleic acid in said mammal.

12. The method of claim 1 1, wherein said second nucleic acid is HCV non-structural protein 3 (NS3).

13. The method of claim 11, wherein said first and second nucleic acid are joined in cis.

14. The method of claim 11, wherein said first and second nucleic acid are juxtaposed.

15. The method of claim 11, wherein said first and second nucleic acid are on the same construct.

16. The method of claim 11, wherein said first and second nucleic acid are on separate constructs.

17. The method of claim 11, wherein said first nucleic acid consists of between 10 and 20 consecutive amino acids of SEQ. ID. NO. 2.

18. The method of claim 11, wherein said first nucleic acid consists of between 20 and 30 consecutive amino acids of SEQ. ID. NO. 2.

19. The method of claim 11, wherein said first nucleic acid consists of between 30 and 40 consecutive amino acids of SEQ. ID. NO. 2.

20. The method of claim 11, wherein said first nucleic acid consists of between 50 and 54 consecutive amino acids of SEQ. ID. NO. 2.


SEQUENCE LISTING (9 sequences)

SEQ ID NO: 1 - protein, 9 aa, Hepatitis C Virus
GAVQNEVTL

SEQ ID NO: 2 - protein, 54 aa, Hepatitis C Virus
STWVLVGGVLAALAAYCLTTGSVVIVGRIILSGKPAIIPDREVLYREFDEMEEC

SEQ ID NO: 3 - DNA, 162 bp, Hepatitis C Virus
agcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgc

SEQ ID NO: 4 - DNA, 30 bp, Hepatitis C Virus
gtggaattcatggcgcctatcacggcctat

SEQ ID NO: 5 - DNA, 24 bp, Hepatitis C Virus
ccacgcggccgcgacgacctacag

SEQ ID NO: 6 - DNA, 30 bp, Hepatitis C Virus
ccctctagatcagcactcttccatttcatc

SEQ ID NO: 7 - DNA, 33 bp, Hepatitis C Virus
ctggaggtcgtcacgcctacctgggtgctcgtt

SEQ ID NO: 8 - DNA, 33 bp, Hepatitis C Virus
aacgagcacccaggtaggcgtgacgacctccag

SEQ ID NO: 9 - protein, 9 aa, Hepatitis C Virus
KAVYNFATM